(19) United States
(12) Patent Application Publication
(10) Pub. No.: US 2026/0253286 A1
(43) Pub. Date: Aug. 27, 2026

(54) SYSTEMS AND METHODS FOR GENERATING COUNTERFACTUAL IMAGES USING COUNTERFACTUAL FINE-TUNING

(71) Applicant: Heartflow, Inc., Mountain View, CA (US)

(72) Inventors: Tian XIA, London (GB); Matthew SINCLAIR, London (GB); Andreas SCHUH, Cambridge (GB); Esther PUYOL-ANTON, London (GB); Samuel GERBER, Fayetteville, WV (US); Peter Kersten PETERSEN, Palo Alto, CA (US); Michiel SCHAAP, Oegstgeest (NL); Benjamin Michael GLOCKER, Cambridge (GB)

(21) Appl. No.: 19/549,396

(22) Filed: Feb. 25, 2026

Related U.S. Application Data
(60) Provisional application No. 63/763,707, filed on Feb. 26, 2025.
(60) Provisional application No. 63/919,092, filed on Nov. 17, 2025.

(51) Int. Cl. G06T 11/60; G06F 40/205; G06T 7/00; G06T 7/10

(52) U.S. Cl. CPC G06T 11/60; G06F 40/205; G06T 7/0012; G06T 7/10; G06T 2200/24; G06T 2207/20081; G06T 2207/20092; G06T 2207/30004; G06T 2210/41

(57) ABSTRACT

A computer-implemented method of training a causal generative model for counterfactual medical image generation includes obtaining medical imaging data including images depicting anatomy of patients and associated causal parameters, obtaining a causal generative model with structural causal functions defining relationships between causal parameters, an encoder network, and a decoder network, and iteratively determining latent parameter noise and a latent representation, obtaining spatially targeted interventions, generating counterfactual parameters and a counterfactual image, determining a loss by applying a pre-trained segmentor to obtain segmentation outputs, determining the loss based on the difference between measurements and counterfactual parameters, and updating the causal generative model. A computer-implemented method of counterfactual image generation may include obtaining medical imaging data including an image depicting patient anatomy, determining latent parameter noise and a latent representation, receiving spatially targeted interventions defining counterfactual changes within regions, generating counterfactual parameters, and generating a counterfactual image by applying a decoder network.

### CROSS-REFERENCES TO RELATED APPLICATIONS

This application claims priority to U.S. Provisional Application No. 63/763,707 filed Feb. 26, 2025, and claims priority to U.S. Provisional Application No. 63/919,092 filed Nov. 17, 2025, the entire disclosures of which are hereby incorporated by reference in their entirety.

### FIELD OF INVENTION

The present disclosure relates to causal generative models for medical image synthesis, and more particularly to systems and methods for generating counterfactual medical images using segmentor-guided counterfactual fine-tuning to enable spatially localized and anatomically coherent modifications of anatomical structures.

### BACKGROUND

Causal generative models have emerged as valuable tools in medical imaging, enabling applications such as data augmentation, bias mitigation, explainability, and disease progression modeling. These models allow researchers and clinicians to explore hypothetical scenarios by generating images that reflect controlled modifications to patient attributes or anatomical structures. For example, such models can simulate how a patient's imaging appearance might change under different clinical conditions, supporting both research and clinical decision-making processes.

Deep Structural Causal Models (DSCMs) represent one approach to integrating causal reasoning with deep generative architectures for high-resolution image synthesis. DSCMs combine structural causal models with hierarchical variational auto-encoders to enable counterfactual image generation, where latent noise is inferred from observed data and interventions are applied to generate modified images. However, standard likelihood-based training of DSCMs can result in suboptimal counterfactual effectiveness, where the generative model may fail to enforce counterfactual consistency and ignore conditioning on intervened-upon parent variables. Counterfactual Fine-Tuning (CFT) using pre-trained regressors or classifiers has been proposed to address this limitation. While regressor-based CFT (Reg-CFT) has demonstrated effectiveness for subject-level interventions such as modifying patient age or sex, it has shown limitations when applied to structure-specific interventions involving localized anatomical regions. Regressors may learn spurious correlations rather than capturing the true spatial semantics of structure-specific variables, resulting in undesirable global changes across the image domain rather than targeted, locally coherent modifications.

This disclosure is directed to various features that address challenges such as one or more of those referenced above. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a computer-implemented method of counterfactual image generation is provided. The method includes obtaining medical imaging data including an image depicting anatomy of a patient. The method includes determining latent parameter noise by applying a learned causal or structural relationship to one or more parameters associated with the medical imaging data. The method includes determining a latent representation by applying an encoder network of a generative model to the image. The method includes receiving one or more spatially targeted interventions, each spatially targeted intervention defining a counterfactual change to be applied to at least one parameter of the one or more parameters within a respective region of the anatomy. The method includes generating a set of counterfactual parameters based on the latent parameter noise and the one or more spatially targeted interventions. The method includes generating a counterfactual image of the anatomy that includes the one or more spatially targeted interventions by applying a decoder network of the generative model to the set of counterfactual parameters and the latent representation.

According to other aspects of the present disclosure, the computer-implemented method of counterfactual image generation may include one or more of the following features. The encoder network may be a Hierarchical Variational Auto Encoder (HVAE) encoder, the decoder network may be a HVAE decoder, and the generative model may be a Deep Structural Causal Model (DSCM) that includes a causal model of the learned causal or structural relationship of the one or more parameters. Determining the latent representation may include applying the encoder network to the image and the one or more parameters, and the generative model may be fine-tuned with segmentation-guided loss to localize counterfactual changes to one or more targeted anatomical regions. The medical imaging data may include at least one of the one or more parameters. The method may further include determining at least one of the one or more parameters by applying an imaging analysis operation to the image. The imaging analysis operation may include segmenting the image using a pre-trained image segmentor. The pre-trained image segmentor may be a same segmentor that was used to train or fine-tune the encoder network and decoder network. Obtaining the medical imaging data may include receiving, via a Graphical User Interface (GUI), a selection of the patient and a selection of the image. Receiving the one or more spatially targeted interventions may include receiving, via the GUI, a natural language prompt describing the counterfactual change to be applied to the image, and parsing the natural language prompt to extract the one or more spatially targeted interventions.

In various embodiments, the causal generative model may be implemented as any suitable causal generative model (e.g., a neural causal model or other structural causal model with learned generative mechanisms), and the DSCM described herein is provided as a non-limiting example.

According to another aspect of the present disclosure, a computer-implemented method of training a causal generative model for counterfactual medical image generation is provided. The method includes obtaining training medical imaging data including images depicting anatomy of respective patients and associated causal parameters. The method includes obtaining a causal generative model that includes (i) structural causal functions defining relationships between the associated causal parameters, and (ii) an encoder network and a decoder network. The method includes iteratively, for one or more images in the training medical imaging data, determining latent parameter noise by applying a current iteration of the structural causal functions to the associated causal parameters, and determining a latent representation by applying a current iteration of the encoder network to an image of the one or more images. The method includes obtaining one or more spatially targeted interventions, each spatially targeted intervention defining a counterfactual change to be applied to at least one parameter of the associated causal parameters within a respective region of the anatomy. The method includes generating a set of counterfactual parameters based on the latent parameter noise and the one or more spatially targeted interventions. The method includes generating a counterfactual image by applying the decoder network to the latent representation and the set of counterfactual parameters. The method includes determining a loss of the generated counterfactual image by applying a pre-trained segmentor to the counterfactual image to obtain segmentation outputs for at least a portion of the anatomy, determining measurements, from the segmentation outputs and corresponding to the set of counterfactual parameters, of the counterfactual image, and determining the loss based on the difference between the measurements and the set of counterfactual parameters. The method includes updating the causal generative model based on the loss.

According to other aspects of the present disclosure, the computer-implemented method of training a causal generative model may include one or more of the following features. Individual parameters of the associated causal parameters may be associated with respective regions of the anatomy, and determining the measurement for an individual parameter may include masking out, weighting, or otherwise restricting any regions of the anatomy not associated with the parameter. Determining the latent representation may include applying the current iteration of the encoder network to the image and the associated causal parameters. The pre-trained segmentor may be frozen during updating of the causal generative model. Obtaining the training medical imaging data may include determining the associated causal parameters by applying the pre-trained segmentor to the images and determining measurements, corresponding to associated parameters, of the images. Obtaining one or more spatially targeted interventions may include selecting a randomized intervention for at least one of the associated causal parameters within a predefined range. Obtaining one or more spatially targeted interventions may include obtaining a distribution of values for at least one of the associated causal parameters and selecting an intervention based on the distribution. The distribution may be based on measurements, corresponding to the at least one of the associated causal parameters, of the images in the training medical imaging data.

According to another aspect of the present disclosure, a system for generating counterfactual images is provided. The system includes at least one memory storing instructions, medical imaging data with an image depicting anatomy of a patient, and a causal generative model that includes a learned causal or structural relationship between parameters associated with the medical imaging data, an encoder network, and a decoder network, wherein the generative model is fine-tuned with segmentation-guided loss to localize counterfactual changes to one or more targeted anatomical regions. The system includes at least one processor operatively connected to the at least one memory and configured to execute the instructions to perform operations. The operations include determining latent parameter noise of the image by applying the learned causal or structural relationship to the parameters. The operations include determining a latent representation by applying the encoder network to the image. The operations include receiving one or more spatially targeted interventions, each spatially targeted intervention defining a counterfactual change to be applied to at least one of the parameters within a respective region of the anatomy. The operations include generating a set of counterfactual parameters based on the latent parameter noise and the one or more spatially targeted interventions. The operations include generating a counterfactual image of the anatomy that includes the one or more spatially targeted interventions by applying the decoder network of the generative model to the set of counterfactual parameters and the latent representation.

According to other aspects of the present disclosure, the system may include one or more of the following features. Determining the latent representation may include applying a Hierarchical Variational Auto Encoder (HVAE) to the image and the parameters. The medical imaging data may include at least one of the parameters. The operations may further include determining at least one of the one or more parameters by applying a pre-trained image segmentor to the image, and the pre-trained image segmentor may be a same segmentor that was used to train the causal generative model.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF FIGURES

Non-limiting and non-exhaustive examples are described with reference to the following figures.

FIG. 1 depicts a block diagram of an exemplary system and network for counterfactual image generation, according to one or more embodiments.

FIG. 2 depicts a process flow diagram of a Deep Structural Causal Model with segmentor-guided counterfactual fine-tuning, according to one or more embodiments.

FIG. 3 depicts a flowchart illustrating method steps for training a causal generative model, according to one or more embodiments.

FIG. 4 depicts a flowchart illustrating method steps for performing counterfactual image generation during inference, according to one or more embodiments.

FIG. 5 depicts visual examples comparing counterfactual images generated using different fine-tuning approaches for chest radiograph interventions, according to one or more embodiments.

FIG. 6 depicts visual results comparing counterfactual images and difference maps generated using different fine-tuning approaches for coronary artery interventions, according to one or more embodiments.

FIG. 7 depicts visual results comparing counterfactual images and difference maps for positional interventions on coronary arteries, according to one or more embodiments.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

Causal generative models for medical imaging face technical challenges when generating counterfactual images that faithfully reflect intended interventions. One such challenge involves ignored counterfactual conditioning, a phenomenon that occurs during standard likelihood-based training of a Hierarchical Variational Auto Encoder (HVAE). During likelihood-based training, the HVAE may learn to ignore conditioning variables, such as anatomical parameters or structure-specific attributes, that are provided as inputs to the decoder. As a result, when a user specifies an intervention on a particular variable (for example, reducing the area of a lung, e.g., in a chest X-ray or the like, or increasing plaque volume, e.g., in a CCTA image, or the like), the generated counterfactual image may fail to reflect the specified change. The HVAE decoder, having learned to disregard the conditioning signal, produces images that do not obey the counterfactual parents, thereby violating counterfactual consistency.

Regressor-based counterfactual fine-tuning (Reg-CFT) represents one approach to address the problem of ignored counterfactual conditioning. In Reg-CFT, pre-trained regressors or classifiers are employed to predict parent variables from generated counterfactual images. The HVAE parameters are then fine-tuned by maximizing the likelihood that the counterfactual parents can be predicted from the generated image, while keeping the regressor weights frozen. This fine-tuning step encourages the DSCM to generate counterfactual images that are consistent with the intended interventions by enforcing that the counterfactual parents are predictable from the generated image.

However, Reg-CFT exhibits limitations when applied to structure-specific interventions, such as modifying the area of an organ or the volume of a localized disease pattern in a medical image. Regressors trained to predict scalar-valued structure-specific variables (for example, left lung area or plaque area) may learn spurious correlations rather than the true spatial semantics of those variables. For instance, a regressor may learn that a particular variable correlates with global image characteristics such as mean pixel intensity, brightness, or texture patterns, rather than with the actual geometric extent of the anatomical structure. When the HVAE is fine-tuned using such a regressor, the DSCM may learn to satisfy the regressor by modifying global image properties rather than by making localized, anatomically coherent changes to the target structure. This results in undesirable global changes across the image domain, including artifacts in regions that were not intended to be modified, rather than targeted modifications confined to the anatomical structure of interest. The regressor-based approach thus provides insufficient guidance for DSCMs to capture the spatial meaning of structure-specific variables, limiting the effectiveness of counterfactual generation for localized anatomical interventions.

Segmentor-guided Counterfactual Fine-Tuning (Seg-CFT) addresses the limitations of regressor-based approaches by leveraging a pre-trained segmentation model during counterfactual fine-tuning. In Seg-CFT, scalar-valued structure-specific variables (for example, left lung area, right lung area, heart area, or plaque area, e.g., for chest X-Ray images) are retained as the causal variables upon which interventions are performed. This retention of scalar-valued variables preserves simplicity of user interaction, as users specify interventions on scalar quantities rather than providing pixel-level segmentation masks or other complex spatial inputs. The pre-trained segmentation model provides spatial grounding during training by producing pixel-level segmentation maps from generated counterfactual images. Measurements corresponding to the scalar-valued structure-specific variables are then derived from the segmentation maps by aggregating pixel-level label probabilities within the relevant anatomical regions. The HVAE parameters are fine-tuned by minimizing the difference between the segmentation-derived measurements and the target counterfactual values specified by the intervention. Because the segmentation model captures the true spatial extent of anatomical structures, the DSCM is forced to produce locally coherent and anatomically meaningful changes that affect the segmentation masks in a manner consistent with the intended intervention. The segmentation model may remain frozen during fine-tuning, and, in some embodiments, counterfactual generation during inference is performed without applying the segmentation model, allowing the generative model to use the same inference-time interface as prior approaches. Although illustrated with a DSCM implementation, Seg-CFT and Pos-Seg-CFT may be applied to other causal generative model architectures configured for counterfactual inference. As used herein, segmentation-guided loss may refer to a loss based on segmentation outputs (optionally converted to measurements) compared to target intervention values.

Positional Segmentor-guided Counterfactual Fine-Tuning (Pos-Seg-CFT) extends Seg-CFT by introducing region-specific supervision that enables spatially targeted interventions. Rather than aggregating measurements across an entire anatomical structure, Pos-Seg-CFT subdivides each structure into regional segments (for example, proximal, mid, and distal regions along a vessel axis from a straightened curved planar reformatted image) and derives independent measurements for each region. Regional measurements are computed by masking out regions not associated with a particular parameter and aggregating pixel-level label probabilities within the region of interest. This decomposition of global structure-specific variables into regional measurements provides position-aware supervision during fine-tuning, enabling explicit control over where within the image the intervention should occur. The same pre-trained segmentation model may be reused for regional measurement extraction by applying spatial masks that partition the image into disjoint regions. Regions may be predefined masks or partitions and need not be output by the segmentor. Pos-Seg-CFT thus enables spatially localized and anatomically coherent counterfactual generation while preserving the simplicity of scalar-valued causal variables and avoiding the need for user-provided pixel-level counterfactual masks.

Counterfactual inference in the context of Seg-CFT and Pos-Seg-CFT follows a three-step process comprising abduction, action, and prediction. In the abduction step, exogenous noise variables are inferred from observed data. For scalar-valued causal variables, abduction involves inverting the learned structural causal functions (implemented as invertible conditional normalizing flows) to recover the latent noise that produced the observed variable values. For the image variable, abduction involves applying the HVAE encoder to the observed image and the causal parent variables to infer the latent image noise. The abducted noise encodes individual-specific characteristics of the patient, such that counterfactual images generated using the same noise differ from the original image due to causal changes rather than due to resampled randomness. In the action step, an intervention is applied by setting one or more causal variables to target counterfactual values using the do-operator. The intervention may specify a change to a global structure-specific variable (as in Seg-CFT) or to a region-specific variable (as in Pos-Seg-CFT). In the prediction step, counterfactual parent values are computed using the abducted noise and the intervened variable values, and the counterfactual image is generated by applying the HVAE decoder to the latent image noise and the counterfactual parent values. The three-step process ensures that the generated counterfactual image reflects the intended intervention while preserving individual-specific characteristics encoded in the abducted noise.

A detailed description of systems, devices, and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

Reference to any particular activity is provided in this disclosure only for convenience and not intended to limit the disclosure. A person of ordinary skill in the art would recognize that the concepts underlying the disclosed devices and methods may be utilized in any suitable activity. For example, while various examples and embodiments may pertain to medical images, techniques according to one or more aspects of this disclosure may be applied to counterfactual generation of images of any subject having parameters that may be modeled via DSCM.

The disclosure may be understood with reference to the following description and the appended drawings, wherein like elements are referred to with the same reference numerals. The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially,” “approximately,” “about,” and “generally,” are used to indicate a possible variation of 10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

In an example of using a conventional DSCM with Reg-CFT, a pre-trained regressor may be employed to predict structure-specific variables from generated counterfactual images. The pre-trained regressor used in Reg-CFT may be a ResNet-based architecture that predicts structure areas from images. For instance, when the DSCM is configured to generate counterfactual chest radiographs with modified lung areas, a ResNet-based regressor may be trained to predict left lung area, right lung area, and heart area from input images. During counterfactual fine-tuning, the DSCM generates a counterfactual image based on an intervention (for example, reducing left lung area by a specified percentage), and the pre-trained regressor predicts the structure areas from the generated counterfactual image. The HVAE parameters are then updated to maximize the likelihood that the regressor predicts the target counterfactual values from the generated image, while the regressor weights remain frozen.

However, the regressor-based approach exhibits failure modes when applied to structure-specific interventions. The ResNet-based regressor, trained to predict scalar-valued structure areas from images, may learn to exploit spurious correlations present in the training data rather than learning the true spatial semantics of the anatomical structures. For example, the regressor may learn that left lung area correlates with global image characteristics such as mean pixel intensity, overall brightness, rib contrast, or texture patterns across the image domain. When the HVAE is fine-tuned using such a regressor, the DSCM may learn to satisfy the regressor by modifying global image properties rather than by making localized changes to the lung boundary. The DSCM may learn that reducing left lung area corresponds to slightly darkening the image or shifting global intensity values, because such global changes cause the regressor to predict a smaller lung area.

This spurious learning results in unintended modifications to non-target structures when intervening on variables like lung area or plaque area. When a user specifies an intervention to reduce left lung area, the DSCM may produce a counterfactual image in which the right lung area and heart area are also modified, even though those structures were not targeted by the intervention. The global intensity changes or texture modifications that satisfy the regressor affect the entire image domain, causing the regressor to predict different values for all structure-specific variables, not just the intervened variable. In the context of coronary artery imaging, interventions on non-calcified plaque area may result in unintended changes to lumen area and calcified plaque area, because the regressor may have learned that plaque area correlates with global vessel brightness or contrast characteristics. The regressor-based approach thus provides insufficient guidance for the DSCM to learn the spatial meaning of structure-specific variables, as the regressor learns what predicts the scalar value rather than where the anatomical structure is located within the image.

In an exemplary use case, training a causal generative model for counterfactual medical image generation according to one or more aspects of this disclosure involves obtaining training medical imaging data including images depicting anatomy of respective patients and associated causal parameters. The training medical imaging data may include medical images such as chest radiographs, coronary computed tomography angiography (CCTA) images, or other imaging modalities that depict anatomical structures of patients. The associated causal parameters may include scalar-valued variables representing structure-specific attributes such as left lung area, right lung area, heart area, calcified plaque area, non-calcified plaque area, lumen area, or other anatomical measurements. The associated causal parameters may be obtained from the training medical imaging data directly (for example, from metadata or annotations accompanying the images) or may be derived by applying imaging analysis operations to the images, such as segmentation followed by measurement extraction.

A Deep Structural Causal Model (DSCM) (or other neural causal model) may be obtained for training. The DSCM includes structural causal functions defining relationships between the associated causal parameters, a Hierarchical Variational Auto Encoder (HVAE) encoder, and an HVAE decoder. The structural causal functions may be implemented as invertible conditional normalizing flows that model the causal relationships between scalar-valued parameters. The HVAE encoder and HVAE decoder together form a generative model capable of encoding images into latent representations and decoding latent representations back into images conditioned on the causal parameters. In various embodiments, a causal generative model may be obtained that includes (i) structural causal functions defining relationships between the associated causal parameters, and (ii) an encoder network and a decoder network. The encoder network may be implemented as an HVAE encoder, and the decoder network may be implemented as an HVAE decoder, though other encoder and decoder architectures are contemplated. The causal generative model may be fine-tuned with segmentation-guided loss to localize counterfactual changes to one or more targeted anatomical regions.

Counterfactual fine-tuning is applied iteratively for individual images in the training medical imaging data. For each iteration, latent parameter noise is determined by applying a current iteration of the structural causal functions to the associated causal parameters. The structural causal functions, being invertible, allow recovery of the exogenous noise variables that produced the observed parameter values. Latent image noise is determined by applying a current iteration of the HVAE encoder to the image. In some embodiments, a latent representation is determined by applying a current iteration of the encoder network to an image of the one or more images in the training medical imaging data. In some cases, determining the latent image noise (or latent representation) includes applying the current iteration of the HVAE encoder (or encoder network) to the image and the associated causal parameters, such that the encoder conditions on both the image and the causal parent variables when inferring the latent representation. As used herein, a “latent representation” may comprise one or more latent variables inferred from an input image by an encoder or inference network, and may be stochastic (e.g., sampled) or deterministic (e.g., an embedding). “Latent parameter noise” may comprise one or more exogenous latent variables corresponding to causal parameters in a structural causal model, and may be inferred during abduction (e.g., by inversion or approximate inference) and used to generate counterfactual parameters under intervention.

One or more spatially targeted interventions are obtained during each iteration. Each spatially targeted intervention defines a counterfactual change to be applied to at least one parameter of the associated parameters within a respective region of the anatomy. For example, a spatially targeted intervention may specify an increase or decrease in plaque area within a proximal, mid, or distal region of a coronary artery, or a change in lung area within a particular anatomical region. The spatially targeted interventions may be obtained by selecting randomized interventions for the associated parameters within predefined ranges, or by sampling intervention values from distributions based on measurements of the images in the training medical imaging data.

A set of counterfactual parameters is generated based on the latent parameter noise and the one or more spatially targeted interventions. The counterfactual parameters reflect the target values specified by the interventions while preserving the latent noise for non-intervened parameters. A counterfactual image is generated by applying the HVAE decoder to the latent image noise and the set of counterfactual parameters. In some embodiments, the counterfactual image is generated by applying the decoder network to the latent representation and the set of counterfactual parameters. The HVAE decoder (or decoder network) produces an image conditioned on the counterfactual parameter values, such that the generated image reflects the intended interventions.

Determining a loss of the generated counterfactual image involves applying a pre-trained segmentor to the counterfactual image to obtain segmentation outputs for at least a portion of the anatomy. The pre-trained segmentor produces pixel-level segmentation maps that delineate anatomical structures and regions within the generated counterfactual image. Measurements corresponding to the set of counterfactual parameters are determined from the counterfactual image from the segmentation outputs and by aggregating pixel-level label probabilities within the relevant anatomical regions identified by the segmentor. For region-specific parameters, measurements may be computed by masking out, weighting, or otherwise restricting regions not associated with a particular parameter and aggregating within the region of interest. The loss is determined based on the difference between the measurements and the set of counterfactual parameters, representing the discrepancy between the segmentation-derived measurements from the generated counterfactual image and the target counterfactual values specified by the interventions.

The DSCM (or causal generative model) is updated based on the loss. The pre-trained segmentor is frozen during updating of the DSCM (or causal generative model), such that gradients flow through the segmentor to update the HVAE encoder and HVAE decoder (or encoder network and decoder network) parameters without modifying the segmentor weights. This frozen segmentor configuration ensures that the segmentor provides consistent spatial supervision throughout the training process. By minimizing the loss based on the difference between segmentation-derived measurements and target counterfactual values, the DSCM learns to produce counterfactual images in which the anatomical structures exhibit the geometric properties specified by the interventions.

Spatially targeted interventions improve the inferences of the trained DSCM by providing position-aware supervision during fine-tuning. When interventions are defined with respect to specific anatomical regions, the DSCM learns to localize changes to the intended regions rather than producing global modifications across the image domain. The region-specific measurements derived from the pre-trained segmentor enforce that changes to a particular anatomical region do not propagate to other regions, reducing off-target effects and improving spatial specificity of the generated counterfactual images. The trained DSCM, having been fine-tuned with spatially targeted interventions, produces counterfactual images that exhibit localized, anatomically coherent modifications aligned with the intended positional interventions.

In another exemplary use case, according to one or more aspects of this disclosure, during inference, counterfactual image generation proceeds through a sequence of operations that transform observed patient data and specified interventions into a counterfactual image reflecting the intended modifications. The inference process leverages a trained DSCM that has been fine-tuned using segmentor-guided counterfactual fine-tuning, as described above. In various embodiments, the inference process may leverage a trained causal generative model that includes an encoder network and a decoder network, wherein the generative model has been fine-tuned with segmentation-guided loss to localize counterfactual changes to one or more targeted anatomical regions.

A computer-implemented method of counterfactual image generation begins with obtaining medical imaging data including an image depicting anatomy of a patient. The medical imaging data may include a chest radiograph, a coronary computed tomography angiography (CCTA) image, or another imaging modality that depicts anatomical structures. The medical imaging data may also include one or more parameters associated with the image, such as scalar-valued structure-specific variables representing anatomical measurements (for example, lung area, heart area, plaque area, or lumen area). In some cases, the one or more parameters may be included directly in the medical imaging data as metadata or annotations. In other cases, the one or more parameters may be derived by applying imaging analysis operations to the image, such as segmentation followed by measurement extraction.

Latent parameter noise is determined by applying a learned causal or structural relationship to the one or more parameters associated with the medical imaging data. The learned causal or structural relationship may be implemented as invertible conditional normalizing flows that model causal dependencies between the parameters. Because the structural causal functions are invertible, the latent parameter noise that produced the observed parameter values can be recovered through inversion of the learned functions. The latent parameter noise encodes individual-specific characteristics of the patient that are not captured by the observed parameter values themselves, representing the exogenous factors that contributed to the patient's particular anatomical configuration.

A latent representation is determined by applying a Hierarchical Variational Auto Encoder (HVAE) encoder (or more generally, an encoder network of a generative model) to the image. The HVAE encoder (or encoder network) maps the observed image into a latent representation that captures patient-specific visual characteristics such as pose, texture, scanner noise, and other appearance factors. In some cases, determining the latent representation includes applying the HVAE encoder (or encoder network) to the image and the one or more parameters, such that the encoder conditions on both the image and the causal parent variables when inferring the latent representation. The latent representation, together with the latent parameter noise, encodes the complete set of exogenous factors that produced the observed patient data. In embodiments where the encoder network is an HVAE encoder and the decoder network is an HVAE decoder, the generative model may be a Deep Structural Causal Model (DSCM) that includes a causal model of the learned causal or structural relationship of the one or more parameters.

One or more spatially targeted interventions are received, each spatially targeted intervention defining a counterfactual change to be applied to at least one parameter of the one or more parameters within a respective region of the anatomy. A spatially targeted intervention may specify, for example, an increase in plaque area within a proximal region of a coronary artery, a decrease in lung area within a particular anatomical region, or a modification to lumen area within a distal vessel segment. The spatially targeted interventions define the counterfactual question being asked, such as “What would this patient's image look like if the plaque area in the mid region were larger?”

A set of counterfactual parameters is generated based on the latent parameter noise and the one or more spatially targeted interventions. For parameters that are subject to intervention, the counterfactual parameter values are set to the target values specified by the interventions. For parameters that are not subject to intervention, the counterfactual parameter values are computed using the latent parameter noise and the learned causal relationships, such that the non-intervened parameters retain values consistent with the same individual patient. The generation of counterfactual parameters preserves the latent parameter noise for non-intervened variables, ensuring that the counterfactual scenario reflects a modification to the same patient rather than a substitution of a different patient.

A counterfactual image of the anatomy that includes the one or more spatially targeted interventions is generated by applying a HVAE decoder (or more generally, a decoder network of the generative model) to the set of counterfactual parameters and the latent representation. The HVAE decoder (or decoder network) produces an image conditioned on the counterfactual parameter values, such that the generated counterfactual image reflects the anatomical modifications specified by the interventions. The counterfactual image generation preserves the same latent noise variables (both the latent parameter noise for non-intervened parameters and the latent representation) to ensure the counterfactual represents the same individual patient rather than a different patient. By holding the latent noise fixed and modifying the causal parameters according to the specified interventions, the generated counterfactual image differs from the original image due to the causal changes rather than due to resampled randomness or substitution of patient identity. The resulting counterfactual image depicts the same patient under the hypothetical scenario defined by the spatially targeted interventions.

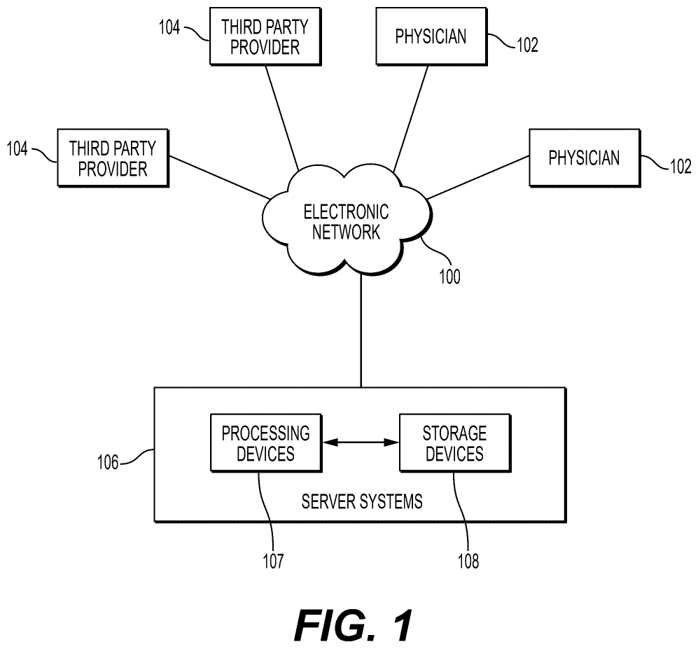

Referring now to the figures, FIG. 1 depicts a block diagram of an exemplary system and network for generating counterfactual medical images. Specifically, FIG. 1 depicts a plurality of physicians 102 and third party providers 104, any of whom may be connected to an electronic network 100, such as the Internet, through one or more computers, servers, and/or handheld mobile devices. Physicians 102 and/or third party providers 104 may create or otherwise obtain images of one or more patients' cardiac and/or vascular systems. The physicians 102 and/or third party providers 104 may also obtain any combination of patient-specific information, such as age, medical history, blood pressure, blood viscosity, etc. Physicians 102 and/or third party providers 104 may transmit the cardiac/vascular images and/or patient-specific information to server systems 106 over the electronic network 100. Server systems 106 may include storage devices for storing images and data received from physicians 102 and/or third party providers 104. Server systems 106 may also include processing devices for processing images and data stored in the storage devices. Alternatively or in addition, the present disclosure (or portions of the system and methods of the present disclosure) may be performed on a local processing device (e.g., a laptop), absent an external server or network.

The storage devices 108 may store medical imaging data of patients, including images depicting anatomy of respective patients and associated parameters. The storage devices 108 may further store training data used for training causal generative models, including training medical imaging data with images and associated causal parameters. The storage devices 108 may also store a DSCM and components of the DSCM. The DSCM stored in the storage devices 108 may include a learned causal or structural relationship between parameters associated with the medical imaging data, a Hierarchical Variational Auto Encoder (HVAE) encoder, and a HVAE decoder. In various embodiments, the storage devices 108 may store a causal generative model that includes structural causal functions defining relationships between the associated causal parameters, an encoder network, and a decoder network. The encoder network may be implemented as an HVAE encoder, and the decoder network may be implemented as an HVAE decoder, though other encoder and decoder architectures are contemplated. The causal generative model may be fine-tuned with segmentation-guided loss to localize counterfactual changes to one or more targeted anatomical regions. The storage devices 108 may additionally store instructions that, when executed by the processing devices 107, cause the processing devices 107 to perform operations for counterfactual image generation.

Medical imaging data may be sourced from devices connected to the electronic network 100. A third party provider 104 may transmit medical imaging data to the server systems 106 over the electronic network 100. The third party provider 104 may include imaging centers, hospitals, clinics, or other healthcare facilities that acquire medical images of patients. A physician 102 may also transmit medical imaging data to the server systems 106 over the electronic network 100. The medical imaging data received from the third party provider 104 or the physician 102 may include images such as chest radiographs, coronary computed tomography angiography (CCTA) images, or other imaging modalities depicting anatomical structures. In some cases, the medical imaging data includes at least one of the parameters associated with the image, such as scalar-valued structure-specific variables representing anatomical measurements that may be provided as metadata or annotations accompanying the images.

The DSCM stored in the storage devices 108 may have an architecture that includes multiple components for causal generative modeling. The learned causal or structural relationship may be implemented as invertible conditional normalizing flows that model causal dependencies between the parameters. The invertible nature of the normalizing flows allows recovery of latent parameter noise from observed parameter values through inversion of the learned functions. The HVAE encoder may map observed images into latent representations that capture patient-specific visual characteristics. The HVAE decoder may produce images conditioned on causal parameter values, generating counterfactual images that reflect anatomical modifications specified by interventions. The DSCM may further include structural causal functions that define relationships between scalar-valued causal parameters, enabling tractable abduction, intervention, and counterfactual reasoning. In some embodiments, the causal generative model stored in the storage devices 108 may include an encoder network and a decoder network that are not necessarily implemented as HVAE components. The encoder network may determine a latent representation by encoding an input image, and the decoder network may generate a counterfactual image by decoding the latent representation conditioned on counterfactual parameters.

The server systems 106 may be accessed via a portal or similar interface from a client device or user device operated by the physician 102 or the third party provider 104. The server systems 106 may provide a Graphical User Interface (GUI) for interacting with the DSCM. The GUI may allow users to select patients and images for counterfactual generation, specify interventions on causal parameters, and view generated counterfactual images. The GUI may display data related to the images, such as identification of tissue or anatomical features, measurements or other anatomical information derived from the images, and risk assessments generated with a risk model. The GUI may present segmentation results showing delineation of anatomical structures within the images, along with scalar-valued measurements corresponding to structure-specific parameters.

The server systems 106 may include a natural language processing (NLP) model to interpret natural language prompts for interventions received via the GUI. A user may enter a natural language prompt describing a counterfactual change to be applied to an image, such as “increase plaque area in the proximal region by 20 percent” or “reduce left lung area by 35 percent.” The NLP model may parse the natural language prompt to extract one or more spatially targeted interventions, each spatially targeted intervention defining a counterfactual change to be applied to at least one of the parameters within a respective region of the anatomy. The extracted interventions may then be provided to the DSCM for counterfactual image generation.

In some embodiments, the DSCM may be trained on a different computer system and subsequently provided to the server systems 106 for deployment. A training system may include specialized hardware such as graphics processing units (GPUs) or tensor processing units (TPUs) configured to accelerate the iterative training process involving the HVAE encoder, HVAE decoder, and structural causal functions. Upon completion of training, the trained DSCM may be transferred to the server systems 106 over the electronic network 100 or through other data transfer mechanisms. The server systems 106 may then store the trained DSCM in the storage devices 108 and execute the DSCM using the processing devices 107 for counterfactual image generation during inference. In various embodiments, instead of residing on the server systems 106, the DSCM may be deployed locally on a user computer device. For example, the DSCM may be integrated or partially integrated into a physician computer operated by the physician 102, a third-party computer operated by the third party provider 104, or another local processing device. Local deployment may enable counterfactual image generation without requiring network connectivity to the server systems 106, which may be advantageous in clinical settings with limited network access or where data privacy considerations favor local processing. A hybrid configuration may also be employed, in which certain components of the DSCM (such as the HVAE encoder) execute locally while other components (such as the HVAE decoder or structural causal functions) execute on the server systems 106.

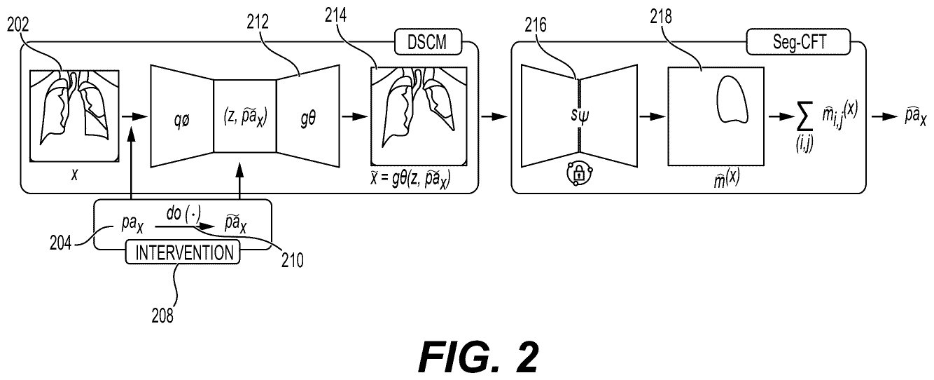

FIG. 2 depicts an exemplary process flow 200 of a DSCM with segmentor-guided counterfactual fine-tuning (Seg-CFT). An input image 202 depicting anatomy of a patient and parameters 204 associated with the input image 202 are provided to an abduction operation 206. The abduction operation 206 infers latent parameter noise from the parameters 204 by inverting the structural causal functions and infers latent image noise from the input image 202 by applying the HVAE encoder. In various embodiments, the abduction operation 206 may infer a latent representation from the input image 202 by applying an encoder network of a generative model. An intervention 208 specifying a counterfactual change to at least one of the parameters 204 is applied at an action operation 210. The action operation 210 generates counterfactual parameters by setting intervened parameters to target values specified by the intervention 208 while computing non-intervened parameters using the abducted latent parameter noise. At a prediction operation 212, a decoder is used to generate a counterfactual image 214 by applying the HVAE decoder to the counterfactual parameters and the latent image noise. In various embodiments, the prediction operation 212 may generate the counterfactual image 214 by applying a decoder network of the generative model to the set of counterfactual parameters and the latent representation. The counterfactual image 214 reflects the anatomical modifications specified by the intervention 208. At a segmentation operation 216 (performed during training and omitted during inference in some embodiments), a pre-trained segmentor is applied to the counterfactual image 214 to generate masks 218. The masks 218 delineate anatomical structures within the counterfactual image 214 and provide pixel-level probability maps for each structure. The masks 218 are used to determine measurements of the anatomical structures in the counterfactual image 214, and an error can be computed between the segmentation-derived measurements and the target counterfactual parameter values specified by the intervention 208. In various embodiments, a loss may be determined based on the difference between the measurements and the set of counterfactual parameters. During training, as discussed in further detail below, error is back-propagated through the segmentor and the decoder to update the DSCM, while the pre-trained segmentor remains frozen such that only the HVAE encoder and HVAE decoder parameters and the structural causal relationships of the parameters are modified during training. In various embodiments, the causal generative model may be updated based on the loss, with the pre-trained segmentor frozen during updating of the causal generative model. Further aspects of these operations are discussed in more detail below.

FIG. 2 illustrates the process flow for Seg-CFT, which operates on global structure-specific parameters. For Positional Segmentor-guided Counterfactual Fine-Tuning (Pos-Seg-CFT), the process flow is modified to incorporate region-specific supervision. In Pos-Seg-CFT, the parameters 204 associated with the input image 202 include region-specific parameters rather than global structure-specific parameters. For example, instead of a single plaque area parameter, the parameters 204 may include separate parameters for plaque area in proximal, mid, and distal regions of a vessel. The intervention 208 in Pos-Seg-CFT specifies counterfactual changes to one or more region-specific parameters, such as increasing plaque area only in the proximal region while leaving mid and distal regions unchanged. At the segmentation operation 216, the masks 218 generated by the pre-trained segmentor are combined with spatial masks that partition the anatomy into disjoint regions. Regional measurements are computed by masking out regions not associated with each parameter and aggregating pixel-level label probabilities only within the region of interest. In various embodiments, determining the measurement for an individual parameter may include masking out, weighting, or otherwise restricting any regions of the anatomy not associated with the parameter. The error in Pos-Seg-CFT is computed as a sum of errors across all regional measurements, comparing each region-specific measurement derived from the counterfactual image 214 against the corresponding target counterfactual value. This region-specific error computation provides position-aware supervision that encourages the DSCM to produce spatially localized modifications confined to the intended anatomical regions without introducing unintended changes in other regions.

The structural causal functions within the DSCM model relationships between scalar causal variables that serve as explicit parents of the image variable. For chest radiograph applications, the scalar causal variables may include age, sex, left lung area (LLA), right lung area (RLA), and heart area (HA). Each of these scalar causal variables may influence the generated image, such that the image variable depends causally on the scalar causal variables according to the structural causal functions. The causal structure may be represented as a directed graph in which age and sex serve as parents of the organ area variables (LLA, RLA, and HA), and the organ area variables in turn serve as parents of the image variable. This causal structure encodes the assumption that patient characteristics such as age and sex influence anatomical measurements, and that the anatomical measurements influence the appearance of the medical image.

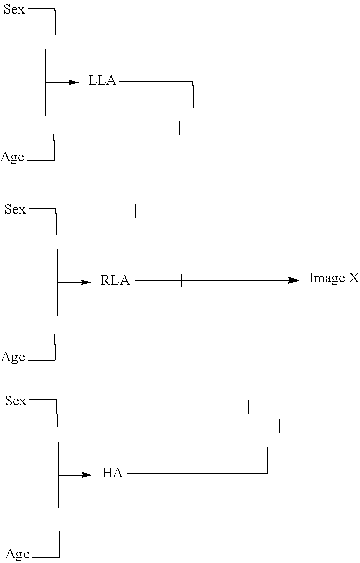

The causal structure depicted above reflects a fundamental modeling assumption in which anatomical measurements such as lung area are causes of the image rather than descriptions derived from the image. This distinction is crucial for proper counterfactual reasoning. In the causal graph, left lung area, right lung area, and heart area are positioned as parents of the image variable, meaning that these anatomical measurements causally influence the appearance of the generated image. The image is not a parent of lung area; rather, lung area is a cause of the image. This causal direction encodes the assumption that a patient's underlying anatomical configuration (including the physical extent of the lungs and heart) determines how the patient appears in a medical image, rather than the image determining the anatomical measurements. If the causal direction were reversed, with the image as a parent of lung area, the model would treat lung area as a derived measurement or description extracted from the image, which would preclude meaningful counterfactual interventions on lung area. By modeling lung area as a causal parent of the image, interventions on lung area propagate forward through the causal graph to produce counterfactual images that reflect the modified anatomical configuration. This causal structure enables the DSCM to answer counterfactual questions such as “What would this patient's chest radiograph look like if the left lung area were smaller?” by intervening on the lung area variable and generating an image consistent with the modified causal parent value.

In another example, for coronary artery disease applications, the structural causal functions may model a different set of scalar causal variables as causal parents of the image. The scalar causal variables for coronary artery imaging may include calcified plaque area (CPA), non-calcified plaque area (NCPA), and lumen area (LA). These variables represent structure-specific attributes of the coronary vasculature that influence the appearance of coronary computed tomography angiography (CCTA) images or straightened curvilinear planar reformation (sCPR) images derived from CCTA data. The causal structure for coronary artery disease modeling may represent CPA, NCPA, and LA as direct parents of the image variable, such that the image depends causally on the plaque and lumen measurements.

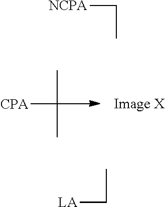

The learned causal or structural relationship between scalar causal variables, such as in the above, is implemented using invertible conditional normalizing flows. A normalizing flow is a generative model that transforms a simple base distribution (such as a standard Gaussian distribution) into a more complex target distribution through a sequence of invertible transformations. In the context of the DSCM, each scalar causal variable is modeled by an invertible conditional normalizing flow that expresses the variable as a function of its parent variables and an exogenous noise variable. For a scalar causal variable aₖ with parents paₖ and exogenous noise uₖ, the structural causal function may be expressed as aₖ=fₖ(uₖ; paₖ), where fₖ is an invertible function implemented by the normalizing flow.

The invertible nature of the normalizing flows enables exact abduction of scalar noise variables through analytical inversion. Given observed values of a scalar causal variable aₖ and its parents paₖ, the exogenous noise uₖ that produced the observed value can be recovered by inverting the structural causal function: uₖ=fₖ⁻¹(aₖ; paₖ). This inversion is exact and deterministic, in contrast to the approximate inference required for high-dimensional variables such as images. The exact abduction of scalar noise variables ensures that counterfactual reasoning for scalar causal variables is tractable and precise, as the latent noise that produced a particular patient's anatomical measurements can be recovered without approximation error.

The causal graph may assume independence between structure-specific variables for simplified modeling. For chest radiograph applications, the causal graph may assume that left lung area, right lung area, and heart area are independent of each other, conditioned on their common parents (such as age and sex). This independence assumption simplifies the causal structure by avoiding direct causal relationships between the organ area variables, such that interventions on one organ area variable do not directly affect other organ area variables through the causal graph. For coronary artery disease applications, the causal graph may similarly assume that calcified plaque area, non-calcified plaque area, and lumen area are independent of each other. This independence assumption is a modeling choice that isolates the effects of interventions on individual structure-specific variables, enabling the DSCM to generate counterfactual images in which changes to one anatomical structure do not propagate to other structures through the causal relationships. The independence assumption may not reflect biological reality in all cases, but provides a tractable framework for counterfactual generation that allows targeted modifications to individual anatomical structures.

The structural causal relationships modeled by the normalizing flows are learned during training rather than being manually specified, predetermined, or implicitly defined. During the training process, the parameters of the invertible conditional normalizing flows are optimized to capture the statistical dependencies present in the training medical imaging data. The training procedure adjusts the flow parameters such that the learned structural causal functions accurately model the conditional distributions of the scalar causal variables given their parent variables. This learning-based approach allows the DSCM to discover causal relationships from data rather than requiring domain experts to manually encode the functional forms of the relationships between variables. The learned structural causal functions may capture nonlinear dependencies and complex distributional characteristics that would be difficult to specify analytically. By learning the causal relationships from training data, the DSCM can adapt to different imaging modalities, patient populations, and anatomical structures without requiring manual reconfiguration of the causal model.

The image variable in the DSCM is modeled using a Hierarchical Variational Auto Encoder (HVAE) rather than an invertible normalizing flow. Unlike scalar causal variables where exact inversion is possible through analytical computation, images are high-dimensional variables for which exact inversion is intractable. The HVAE provides an approximate inference mechanism that enables abduction of latent image noise from observed images while maintaining the causal structure required for counterfactual generation.

Continuing the chest radiograph example, the image variable X depends causally on the same parent variables that influence the scalar causal variables. The image X is a function of the organ area variables (left lung area, right lung area, and heart area), the patient characteristics (age and sex), and a latent noise vector z that captures patient-specific visual characteristics not explained by the causal parents. The latent noise vector z encodes individual-specific appearance factors such as pose, body habitus, scanner characteristics, image contrast, and other visual properties that vary across patients independently of the causal parent variables.

The HVAE encoder approximates the posterior distribution over the latent noise vector z given the observed image and the causal parent variables. The encoder may be expressed as:

$${q}_{ϕ} ( z \vert x , {pa}_{x} ) \approx p ( z \vert x , {pa}_{x} )$$

where q_{φ} denotes the approximate posterior parameterized by encoder parameters φ, x denotes the observed image, and paₓ denotes the causal parent variables of the image (such as left lung area, right lung area, heart area, age, and sex). The encoder takes as input both the observed image and the causal parent values, producing a distribution over the latent noise vector z. During abduction, a sample from this approximate posterior serves as the inferred latent image noise for the observed patient.

The HVAE decoder generates images conditioned on the latent noise vector and the causal parent variables. The decoder may be expressed as:

$${p}_{θ} ( x \vert z , {pa}_{x} )$$

where p_{θ} denotes the conditional likelihood parameterized by decoder parameters θ. The decoder takes as input the latent noise vector z and the causal parent values paₓ, producing a distribution over images. During counterfactual generation, the decoder generates a counterfactual image by conditioning on the abducted latent noise z and the counterfactual parent values specified by the intervention.

This architecture allows the image to depend causally on the same parents as the scalar causal variables (lung area, age, sex, and other anatomical measurements) while also depending on a latent noise vector z that captures individual-specific visual characteristics. The causal dependence on parent variables ensures that interventions on those variables propagate to the generated image, while the dependence on the latent noise vector ensures that counterfactual images preserve patient-specific appearance factors that are independent of the intervention.

As discussed above, the DSCM may include a pre-trained segmentor. The segmentor may be used, in Pos-Seg-CFT applications, to define regions of an input image and take measurements, and in both Pos-Seg-CFT and Seg-CFT to take region-specific measurements during fine-tuning in the training process. The pre-trained segmentor produces probability maps for multiple anatomical structures. To continue the chest radiograph applications, the pre-trained segmentor may produce probability maps for left lung, right lung, and heart regions. Each probability map assigns a probability value between zero and one to each pixel, indicating the likelihood that the pixel belongs to the corresponding anatomical structure. The probability maps may be interpreted as soft segmentation masks that capture uncertainty in structure boundaries while providing continuous values suitable for gradient-based optimization during counterfactual fine-tuning. For instance, the left lung probability map delineates the spatial extent of the left lung within the chest radiograph, the right lung probability map delineates the spatial extent of the right lung, and the heart probability map delineates the cardiac silhouette.

For coronary artery imaging applications, the pre-trained segmentor may produce probability maps for coronary structures including lumen, calcified plaque, and non-calcified plaque. The lumen probability map identifies pixels corresponding to the vessel lumen through which blood flows. The calcified plaque probability map identifies pixels corresponding to calcified atherosclerotic deposits within the vessel wall. The non-calcified plaque probability map identifies pixels corresponding to non-calcified (soft) plaque components. These probability maps enable measurement extraction for structure-specific variables such as lumen area, calcified plaque area, and non-calcified plaque area by aggregating pixel-level probabilities within the relevant anatomical regions.

As described previously, the pre-trained segmentor is frozen during updating of the DSCM. The frozen configuration of the pre-trained segmentor ensures that the segmentor weights remain fixed throughout the counterfactual fine-tuning process. This frozen segmentor configuration provides consistent spatial supervision throughout training, as the segmentor produces stable and reproducible segmentation outputs that do not drift or change as the DSCM parameters are updated. The frozen segmentor serves as a fixed reference for measuring anatomical structure areas, ensuring that the DSCM learns to produce counterfactual images that satisfy the segmentation-derived measurements according to a consistent standard.

In some embodiments, the segmentor used for evaluating counterfactual effectiveness during testing is trained independently from the segmentor used during fine-tuning. This independent training of the evaluation segmentor ensures unbiased assessment of counterfactual generation quality. If the same segmentor were used for both fine-tuning and evaluation, the DSCM might learn to exploit specific characteristics or biases of that particular segmentor rather than learning to produce genuinely anatomically coherent counterfactual images. By training a separate evaluation segmentor on the same segmentation task but with different random initialization or training data splits, the evaluation process measures whether the generated counterfactual images exhibit the intended anatomical modifications according to an independent assessment criterion. The independently trained evaluation segmentor provides an unbiased measure of whether the DSCM has learned to produce counterfactual images with correct anatomical structure areas, rather than merely learning to satisfy the particular segmentor used during fine-tuning.

Regional measurements in Pos-Seg-CFT are computed from segmentation outputs by aggregating pixel-level label probabilities within defined anatomical regions. The measurements corresponding to counterfactual parameters are computed by summing pixel-level label probabilities from the segmentation maps produced by the pre-trained segmentor. For a given anatomical structure, the segmentation map assigns a probability value to each pixel indicating the likelihood that the pixel belongs to that structure. The measurement for the structure is obtained by summing these probability values across all pixels within the relevant region, yielding a scalar value representing the area of the structure within that region.

Individual parameters of the associated parameters are associated with respective regions of the anatomy. For the coronary artery imaging application above, the respective regions of the anatomy may be defined as proximal, mid, and distal segments along a vessel axis. These regional definitions partition the vessel into anatomically meaningful segments that correspond to clinical conventions for describing coronary artery anatomy. A parameter such as calcified plaque area in the proximal region represents the area of calcified plaque within the proximal segment of the vessel, while calcified plaque area in the mid region represents the area within the mid segment, and calcified plaque area in the distal region represents the area within the distal segment. Similarly, non-calcified plaque area and lumen area may each be associated with proximal, mid, and distal regions, yielding region-specific parameters for each anatomical structure.

Determining the measurement for an individual parameter includes masking out any regions of the anatomy not associated with the parameter. Spatial masks defining regions partition the image into disjoint regions that together cover the entire anatomical structure of interest. For coronary artery imaging, a set of spatial masks may define the proximal, mid, and distal regions respectively, where each mask identifies the pixel locations belonging to that region. The spatial masks are disjoint, meaning that no pixel belongs to more than one region, and together the masks cover the entire vessel segment of interest. When computing the regional measurement for a parameter associated with a particular region, the spatial mask for that region is applied to the segmentation map, and pixels outside the region are masked out (excluded from the summation). The regional measurement is then computed by summing the pixel-level label probabilities only within the region of interest.

The regional measurements are computed by reusing the same pre-trained segmentor while masking out other regions and aggregating within the region of interest. The pre-trained segmentor produces a segmentation map for the entire image, identifying the spatial extent of each anatomical structure across all regions. To obtain a regional measurement, the segmentation map is combined with the spatial mask for the region of interest. Pixels outside the region of interest are masked out by setting their contribution to zero, and the remaining pixel-level label probabilities within the region are summed to yield the regional measurement. This approach allows a single pre-trained segmentor to provide measurements for multiple region-specific parameters without requiring separate segmentors for each region. The same segmentor that produces the full segmentation map is reused for all regional measurements, with the spatial masks determining which pixels contribute to each measurement.

Spatially targeted interventions may be specified using different mathematical formulations depending on the nature of the counterfactual change to be applied. A spatially targeted intervention may specify a multiplicative scaling factor applied to a structure-specific parameter. For example, a multiplicative intervention may reduce left lung area by 35 percent. Multiplicative scaling factors allow interventions to be expressed as proportional changes relative to the observed value, which may be useful when the magnitude of the change should scale with the size of the anatomical structure.

Alternatively, a spatially targeted intervention may specify an additive change to a structure-specific parameter. For example, an additive intervention may increase calcified plaque area by 20 square millimeters, which corresponds to adding 20 square millimeters to the observed calcified plaque area value regardless of the original measurement. Additive interventions allow changes to be expressed in absolute units, which may be useful when the clinical significance of the change is defined in terms of absolute quantities rather than proportional changes.

During training, obtaining one or more spatially targeted interventions may include selecting a randomized intervention for at least one of the associated parameters within a predefined range. Obtaining one or more spatially targeted interventions may also include obtaining a distribution of values for at least one of the associated parameters and selecting an intervention based on the distribution. The distribution may be based on measurements, corresponding to the at least one of the associated parameters, of the images in the training medical imaging data. For example, if the training medical imaging data includes measurements of left lung area for a population of patients, the distribution of left lung area values across the training population may be computed. An intervention target value may then be sampled from this distribution, ensuring that the counterfactual target values encountered during training reflect the range of anatomically plausible values observed in the patient population. This distribution-based sampling approach ensures that the DSCM is trained on intervention targets that fall within the range of values present in the training data.

The interventions used during training affect the scope of interventions that may be applied during inference. The DSCM learns a mapping from causal parameter values and latent noise to generated images, and this mapping is learned from the training data. The scope of valid interventions during inference is constrained by several factors related to the training process.

A first constraint relates to which variables exist in the causal graph. Interventions may be applied during inference to variables that are explicitly modeled in the causal graph, have associated structural equations implemented as normalizing flows, and are included as conditioning variables for the HVAE. Variables that are not modeled in the causal graph cannot be intervened upon during inference. For example, if the causal graph for chest radiograph generation includes sex, age, left lung area, right lung area, and heart area as causal variables, interventions may be applied to any of these variables. However, interventions on variables such as rib texture, scanner model, or tumor shape cannot be performed unless those variables are explicitly modeled in the causal graph with associated structural equations.

A second constraint relates to which variables were used during counterfactual fine-tuning. Even if a variable exists in the causal graph, the variable may be included in HVAE conditioning and used in the counterfactual fine-tuning loss function for the DSCM to learn to respond to interventions on that variable. If a variable is included in the causal graph but is not used during counterfactual fine-tuning, the HVAE decoder may learn to ignore conditioning on that variable, resulting in weak or absent control over that variable during inference. Variables that appeared in the counterfactual fine-tuning training process serve as active controls during inference, while variables that were not included in fine-tuning may exhibit reduced responsiveness to interventions.

A third constraint relates to the value ranges seen during training. The DSCM learns meaningful responses to interventions within the range of parameter values encountered during training. If the training data includes left lung area values in the range of 7,000 to 11,000 square pixels, the DSCM learns to generate anatomically coherent images for counterfactual target values within or near this range. Interventions specifying target values far outside the training distribution may produce unreliable results, including anatomical inconsistencies or image artifacts, because the DSCM is extrapolating beyond the learned manifold.

Segmentor-guided counterfactual fine-tuning strengthens the relationship between intervention semantics and the training process. Because Seg-CFT teaches the DSCM the geometric meaning of structure-specific variables through segmentation-derived measurements, the DSCM learns what changing a particular parameter means in terms of spatial modifications to anatomical structures. The segmentation-based supervision teaches the DSCM the location and boundaries of anatomical structures, ensuring that interventions produce spatially coherent changes. However, this geometric understanding is learned for the structures and parameter ranges present in the training data, and the DSCM may not generalize to structures or parameter ranges not encountered during training.

Interventions during inference are not limited to the exact discrete values used during training. Because the DSCM is implemented using neural networks that learn continuous mappings, the model may interpolate between training values to generate counterfactual images for intervention targets not explicitly seen during training. If the training process samples multipliers of 0.8, 1.0, and 1.2, the DSCM may generate reasonable counterfactual images for intervention multipliers of 0.9 or 1.1 through interpolation. The continuous nature of the learned mapping allows interventions to be specified at any value within the learned manifold, rather than being restricted to the exact discrete values sampled during training.

The DSCM includes a do-operator module that implements interventions on causal variables. The do-operator module receives a specification of which variables are to be intervened upon and the target values for those variables. When an intervention do(aₖ=aₖ) is applied, the do-operator module sets the value of the intervened variable aₖ to the specified target value aₖ while disconnecting that variable from its causal parents in the graph. This disconnection ensures that the intervened variable takes the specified value regardless of what its parent variables would have implied, reflecting the causal semantics of an intervention rather than mere conditioning. For non-intervened variables, the do-operator module preserves the abducted exogenous noise and applies the learned structural causal functions to compute counterfactual values consistent with the intervention. The do-operator module thus transforms the abducted noise variables and intervention specifications into a complete set of counterfactual parameter values that can be passed to the decoder for image generation.

The HVAE decoder serves as the image generation module of the DSCM. The decoder receives the counterfactual parameter values produced by the do-operator module along with the abducted latent image noise z. The decoder architecture may comprise a hierarchical structure with multiple resolution levels, enabling generation of high-resolution medical images with fine anatomical detail. At each level of the hierarchy, the decoder conditions on the causal parent variables, allowing the anatomical measurements to influence the generated image at multiple spatial scales. The decoder produces an output image that reflects both the counterfactual parameter values (encoding the intended anatomical modifications) and the latent image noise (encoding patient-specific visual characteristics). The conditioning mechanism within the decoder may be implemented through concatenation, feature-wise linear modulation, or other conditioning architectures that allow the causal parent values to modulate the image generation process.

During training, the counterfactual image may be evaluated against the counterfactual parameters resulting from the do-operator module. The error between measurements and counterfactual parameters may be computed using an L1 loss function or an L2 loss function. An L1 loss function computes the absolute difference between the predicted measurement and the target counterfactual value, while an L2 loss function computes the squared difference between the predicted measurement and the target counterfactual value. The L1 loss function may be expressed as:

$${ℒ}_{L 1} = \vert \hat{{pa}_{x}} - \tilde{{pa}_{x}} \vert$$

where paₓ denotes the measurement derived from the segmentation of the generated counterfactual image and paₓ denotes the target counterfactual value specified by the intervention. The L2 loss function may be expressed as:

$${ℒ}_{L 2} = {( \hat{{pa}_{x}} - \tilde{{pa}_{x}} )}^{2}$$

The choice between L1 and L2 loss functions may depend on the desired sensitivity to outliers and the optimization characteristics for a particular application. The L1 loss function provides robustness to outliers by penalizing errors linearly, while the L2 loss function penalizes larger errors more heavily due to the quadratic term.

For Seg-CFT, the loss function computes the error between the segmentation-derived measurement of the entire anatomical structure and the target counterfactual value. The Seg-CFT loss function may be expressed as:

$${ℒ}_{𝒮ℯℊ - 𝒞ℱ𝒯} = ℓ ( \hat{{pa}_{x}} , \tilde{{pa}_{x}} )$$

where e denotes a scalar loss function such as the L1 or L2 loss. The measurement \hat{p}aₓ is computed by summing pixel-level label probabilities from the segmentation map produced by the pre-trained segmentor across the entire anatomical structure of interest. This global measurement captures the total area of the structure without distinguishing between different spatial regions within the structure.

For Pos-Seg-CFT, the loss function sums losses across regional measurements to provide position-aware supervision. The Pos-Seg-CFT loss function may be expressed as:

$${ℒ}_{𝒫ℴ𝓈 - 𝒮ℯℊ - 𝒞ℱ𝒯} ( θ , ϕ ) = {\sum }_{k = 1}^{K} ℒ ( {\hat{pa}}_{x}^{( k )} , {\tilde{pa}}_{x}^{( k )} )$$

where K denotes the number of disjoint regions into which the anatomical structure is partitioned,

$${\hat{pa}}_{x}^{( k )}$$

denotes the predicted measurement within region k derived from the segmentation of the generated counterfactual image, and

$${\tilde{pa}}_{x}^{( k )}$$

denotes the target counterfactual measurement for region k. The regional measurement

$${\hat{pa}}_{x}^{( k )}$$

is computed by summing pixel-level label probabilities only within the spatial extent of region k, with pixels outside region k masked out and excluded from the summation. The summation of losses across all K regions ensures that the DSCM receives supervision for each regional measurement independently, encouraging the model to produce spatially localized modifications that satisfy the target values within each respective region without introducing unintended changes in other regions. In various embodiments, the causal generative model may be updated based on the loss, wherein the loss is determined based on the difference between the measurements and the set of counterfactual parameters.

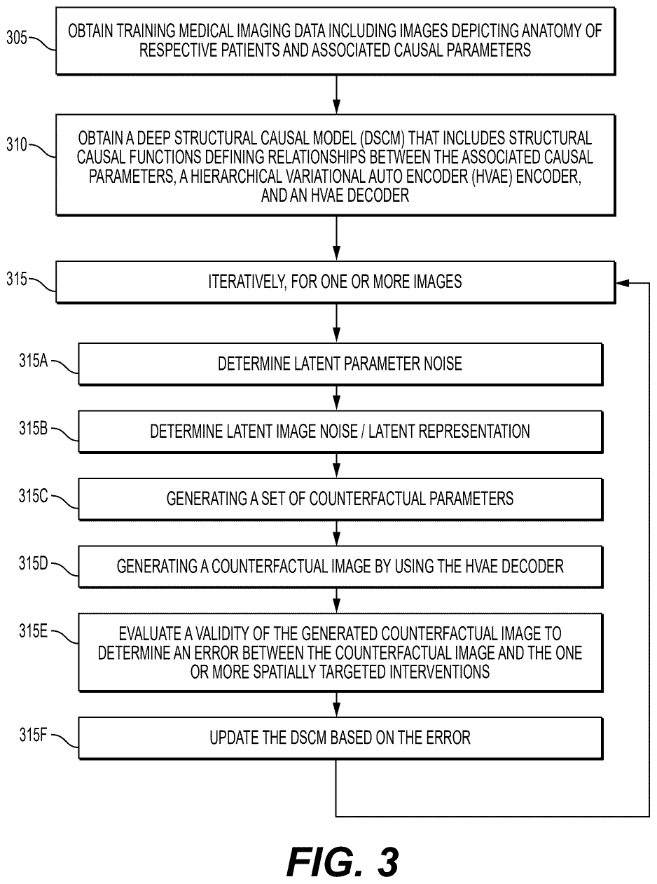

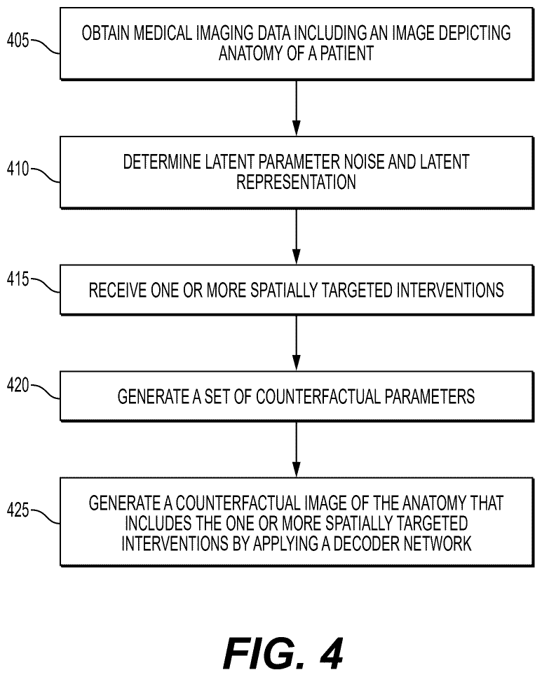

Referring now to FIGS. 3-4, flowcharts illustrating method steps for training a causal generative model and for performing counterfactual image generation during inference are depicted. FIG. 3 illustrates a training process for a DSCM, while FIG. 4 illustrates an inference process for generating counterfactual images using a trained DSCM. The HVAE encoder and HVAE decoder are parts of the DSCM that includes a causal model of the learned causal or structural relationship of the one or more parameters, as described previously. In various embodiments, the encoder network may be a Hierarchical Variational Auto Encoder (HVAE) encoder, the decoder network may be a HVAE decoder, and the generative model may be a Deep Structural Causal Model (DSCM) that includes a causal model of the learned causal or structural relationship of the one or more parameters.

With reference to FIG. 3, a step 305 includes obtaining training medical imaging data including images depicting anatomy of respective patients and associated causal parameters. The training medical imaging data may be retrieved from the storage devices 108 of the server systems 106 or received from the physician 102 or the third party provider 104 over the electronic network 100. The training medical imaging data may include medical images such as chest radiographs, coronary computed tomography angiography (CCTA) images, or other imaging modalities depicting anatomical structures. The associated causal parameters may include scalar-valued variables representing structure-specific attributes such as lung area, heart area, plaque area, or lumen area. In some cases, the training medical imaging data includes at least one of the associated causal parameters as metadata or annotations accompanying the images. In other cases, at least one of the associated causal parameters may be determined by applying an imaging analysis operation to the images. The imaging analysis operation may include segmenting the images using a pre-trained image segmentor to identify anatomical structures and derive measurements corresponding to the associated causal parameters.

A step 310 includes obtaining a DSCM that includes structural causal functions defining relationships between the associated causal parameters, a HVAE encoder, and an HVAE decoder. The DSCM may be retrieved from the storage devices 108 or initialized with random or pre-trained parameters. The structural causal functions may be implemented as invertible conditional normalizing flows that model causal dependencies between the scalar-valued parameters. The HVAE encoder and HVAE decoder together form a generative model capable of encoding images into latent representations and decoding latent representations back into images conditioned on the causal parameters. In various embodiments, step 310 may include obtaining a causal generative model that includes (i) structural causal functions defining relationships between the associated causal parameters, and (ii) an encoder network and a decoder network. The generative model may be fine-tuned with segmentation-guided loss to localize counterfactual changes to one or more targeted anatomical regions.

A step 315 includes iteratively processing individual images in the training medical imaging data to perform counterfactual fine-tuning. The iterative processing includes multiple sub-steps that are performed for each image in the training data. In various embodiments, step 315 includes iteratively processing one or more images in the training medical imaging data. A step 315A includes determining latent parameter noise by applying a current iteration of the structural causal functions to the associated causal parameters.

A step 315B includes determining latent image noise by applying a current iteration of the HVAE encoder to the image. Determining the latent image noise may include applying the HVAE encoder to the image and the one or more parameters, such that the encoder conditions on both the image and the causal parent variables when inferring the latent representation. This conditioning allows the encoder to produce latent representations that account for the causal structure of the model. In various embodiments, step 315B includes determining a latent representation by applying a current iteration of the encoder network to an image of the one or more images.

A step 315C includes obtaining one or more spatially targeted interventions, each spatially targeted intervention defining a counterfactual change to be applied to at least one parameter of the associated parameters within a respective region of the anatomy. The spatially targeted interventions may be obtained by selecting randomized interventions for the associated parameters within predefined ranges, or by sampling intervention values from distributions based on measurements of the images in the training medical imaging data. In various embodiments, each spatially targeted intervention defines a counterfactual change to be applied to at least one parameter of the associated causal parameters within a respective region of the anatomy.

A step 315D includes generating a set of counterfactual parameters based on the latent parameter noise and the one or more spatially targeted interventions. For parameters subject to intervention, the counterfactual parameter values are set to the target values specified by the interventions. For parameters not subject to intervention, the counterfactual parameter values are computed using the latent parameter noise and the learned causal relationships.

A step 315E includes generating a counterfactual image by applying the HVAE decoder to the latent image noise and the set of counterfactual parameters. The HVAE decoder produces an image conditioned on the counterfactual parameter values, such that the generated image reflects the intended interventions. In various embodiments, step 315E includes generating a counterfactual image by applying the decoder network to the latent representation and the set of counterfactual parameters.

A step 315F includes evaluating a validity of the generated counterfactual image. In various embodiments, step 315F includes determining a loss of the generated counterfactual image. Evaluating the validity includes applying a pre-trained segmentor to the counterfactual image to identify the respective regions of the anatomy. In various embodiments, determining a loss may include applying a pre-trained segmentor to the counterfactual image to obtain segmentation outputs for at least a portion of the anatomy. The pre-trained segmentor produces pixel-level segmentation maps that delineate anatomical structures and regions within the generated counterfactual image. Measurements corresponding to the set of counterfactual parameters are determined from the counterfactual image by aggregating pixel-level label probabilities within the relevant anatomical regions identified by the segmentor. In various embodiments, measurements are determined from the segmentation outputs and corresponding to the set of counterfactual parameters. An error between the measurements and the set of counterfactual parameters is determined, representing the discrepancy between the segmentation-derived measurements and the target counterfactual values. In various embodiments, the loss is determined based on the difference between the measurements and the set of counterfactual parameters. The pre-trained image segmentor used during training may be the same segmentor that was used to extract the parameters from the medical imaging data.

A step 320 includes updating the DSCM based on the error. In various embodiments, step 320 includes updating the causal generative model based on the loss. The pre-trained segmentor is frozen during updating of the DSCM, such that gradients flow through the segmentor to update the HVAE encoder and HVAE decoder parameters without modifying the segmentor weights. In various embodiments, the pre-trained segmentor is frozen during updating of the causal generative model. The iterative processing of step 315 and the updating of step 320 are repeated for multiple images and multiple training iterations until the DSCM converges to produce counterfactual images that satisfy the segmentation-derived measurements.

Referring now to FIG. 4, the inference process for counterfactual image generation using a trained DSCM is illustrated. A step 405 includes obtaining medical imaging data including an image depicting anatomy of a patient. The medical imaging data may be retrieved from the storage devices 108 or received from the physician 102 or the third party provider 104 over the electronic network 100. Obtaining the medical imaging data may include receiving, via a Graphical User Interface (GUI), a selection of the patient and a selection of the image. The GUI may be provided by the server systems 106 and accessed via a client device operated by the physician 102 or the third party provider 104. The medical imaging data may include at least one of the one or more parameters associated with the image, such as scalar-valued structure-specific variables provided as metadata or annotations. In some cases, at least one of the one or more parameters may be determined by applying an imaging analysis operation to the image. The imaging analysis operation may include segmenting the image using a pre-trained image segmentor to identify anatomical structures and derive measurements. The pre-trained image segmentor may be the same segmentor that was used to train the HVAE encoder and HVAE decoder, ensuring consistency between training and inference.

A step 410 includes determining latent parameter noise by applying a learned causal or structural relationship to the one or more parameters associated with the medical imaging data. A step 415 includes determining latent image noise by applying the HVAE encoder to the image. Determining the latent image noise may include applying the HVAE encoder to the image and the one or more parameters, such that the encoder conditions on both the image and the causal parent variables when inferring the latent representation. In various embodiments, step 415 includes determining a latent representation by applying an encoder network of a generative model to the image. In various embodiments, determining the latent representation includes applying the encoder network to the image and the one or more parameters, and the generative model is fine-tuned with segmentation-guided loss to localize counterfactual changes to one or more targeted anatomical regions.

A step 420 includes receiving one or more spatially targeted interventions, each spatially targeted intervention defining a counterfactual change to be applied to at least one parameter of the one or more parameters within a respective region of the anatomy. Receiving the one or more spatially targeted interventions may include receiving, via the GUI, a natural language prompt describing the counterfactual change to be applied to the image. The natural language prompt may describe the intended modification in terms understandable to a clinician, such as “increase plaque area in the proximal region by 20 percent” or “reduce left lung area by 35 percent.” Receiving the one or more spatially targeted interventions may further include parsing the natural language prompt to extract the one or more spatially targeted interventions. The parsing may be performed by a natural language processing (NLP) model that identifies the target parameter, the region of the anatomy, and the magnitude of the counterfactual change from the natural language prompt. As used herein, a “spatially targeted intervention” may refer to (i) a target parameter, (ii) a target region, and/or (iii) a target value or change in magnitude for the target parameter, wherein one or more of (i)-(iii) may be explicitly provided or derived. A “region” may be defined as an anatomical subregion, a partition of an anatomical structure, a mask (binary or weighted), a coordinate range, or a segment along a centerline or other reference path.

A step 425 includes generating a set of counterfactual parameters based on the latent parameter noise and the one or more spatially targeted interventions, and generating a counterfactual image of the anatomy that includes the one or more spatially targeted interventions by applying the HVAE decoder to the set of counterfactual parameters and the latent image noise. The HVAE decoder produces an image conditioned on the counterfactual parameter values, such that the generated counterfactual image reflects the anatomical modifications specified by the interventions while preserving patient-specific characteristics encoded in the latent noise. In various embodiments, step 425 includes generating a counterfactual image of the anatomy that includes the one or more spatially targeted interventions by applying a decoder network of the generative model to the set of counterfactual parameters and the latent representation. The generated counterfactual image may be displayed via the GUI or stored in the storage devices 108 for subsequent review by the physician 102 or the third party provider 104.

Experiments were conducted on two datasets: the publicly available PadChest and an internal coronary computed tomography angiography (CCTA) dataset. The evaluation compares the proposed Seg-CFT with Reg-CFT for structure-specific interventions, in particular by modifying the areas of targeted structures. For Reg-CFT, ResNet-based regressors were pre-trained to predict the area of structures. For Seg-CFT, U-Net segmentors were pre-trained using a Dice loss to produce 2D label maps. Counterfactuals were evaluated via effectiveness, which assesses whether generated images obey the counterfactual parents.

Evaluation of the chest radiographs from the PadChest dataset, intervening on the size of anatomical structures. 85 k subjects were manually selected around, removing mislabeled or low-quality images. The resulting dataset consists of 61,714 images for training, 6,911 for validation and 17,123 for testing. All images were resized to 224×224 pixels. Three structure-specific variables were considered: left lung area (LLA), right lung area (RLA) and heart area (HA). For simplicity, it was assumed that they are independent of each other. The original PadChest data does not have segmentation masks. Masks obtained with the pre-trained segmentation model of the torchxrayvison package for left lung, right lung, and heart, respectively were used.

An end-to-end example illustrates the counterfactual generation process for the chest radiograph intervention. The example demonstrates an intervention of shrinking the left lung by 35 percent while keeping all other anatomical structures unchanged. As discussed above, the DSCM for this application includes causal variables comprising sex (a binary variable serving as a parent of organ area variables), age (a scalar variable serving as a parent of organ area variables), left lung area (a scalar variable that is the intervention target), right lung area (a scalar variable that is not intervened upon), heart area (a scalar variable that is not intervened upon), and the image (a high-dimensional variable that is generated by the DSCM).

To run through an iteration of the training process for this evaluation, an observed patient presents with a real chest radiograph image having known attributes. The observed values for this patient include sex of male, age of 60 years, left lung area of 9,368 square pixels, right lung area of 8,291 square pixels, and heart area of 3,476 square pixels. The image depicts a chest radiograph of this patient.

The abduction step infers the hidden noise that produced the observed patient data. For scalar variables, exact inversion through the normalizing flows recovers the exogenous noise values. Notional noise values for this example include u_LLA of +0.41, u_RLA of −0.12, and u_HA of +0.05. These noise values encode individual-specific anatomy of the patient. For the image latent, the HVAE encoder produces an approximate inference of the latent image noise z. A notional latent vector z=[0.87, −1.22, 0.14, . . . ] captures patient-specific characteristics including pose, texture, scanner noise, and other appearance factors.

The action step applies a causal intervention to answer the counterfactual question: “What if the left lung area were 35 percent smaller?” The new target value is computed as 0.65×9,368=6,089 square pixels. The intervention operator do(LLA=6089) sets the left lung area to the target value while all other noise terms remain identical to the abducted values.

The prediction step computes counterfactual parent values using the same abducted noise. The left lung area is fixed by the do-operator at 6,089 square pixels. The right lung area is recomputed using the structural causal function f(u_RLA; Sex, Age) with the abducted noise u_RLA, yielding approximately 8,291 square pixels. The heart area is similarly recomputed using f(u_HA; Sex, Age) with the abducted noise u_HA, yielding approximately 3,476 square pixels. The right lung area and heart area are recomputed rather than copied, but remain unchanged because the noise values are unchanged and no intervention was applied to those variables.

The DSCM decoder generates a counterfactual image conditioned on the counterfactual parent values and the latent image noise z. Without Seg-CFT training, the model may exhibit failure modes including subtly darkening the image, shifting contrast globally, or barely changing lung geometry. These failure modes arise because the HVAE decoder, trained with likelihood-based objectives, may learn to ignore the conditioning variables or satisfy regressor-based supervision through spurious correlations rather than anatomically meaningful changes.

During training with Seg-CFT, the frozen segmentor is applied to the generated counterfactual image. The segmentor produces probability masks for the left lung, right lung, and heart, with each pixel assigned a probability value between zero and one. The actual lung area is measured from the segmentation by summing pixel-level label probabilities. If the model produces a counterfactual image with predicted left lung area of 8,543 square pixels (rather than the target of 6,089 square pixels), the Seg-CFT loss is computed as the absolute difference: |8,543-6,089|=2,454 square pixels. The gradient from this loss flows through the frozen segmentor back into the DSCM decoder, penalizing the model for producing a lung that is too large in the spatial region where the lung is located.

Through iterative training with Seg-CFT, the DSCM learns to produce anatomically coherent counterfactual images. Solutions that Reg-CFT may allow but that Seg-CFT prevents include global intensity changes, texture artifacts, and unintended shrinking of the heart or right lung. The segmentation-based supervision enforces that the DSCM learns to move the lung boundary inward, reduce lung pixel mass within the left lung region, and preserve the heart and right lung unchanged.

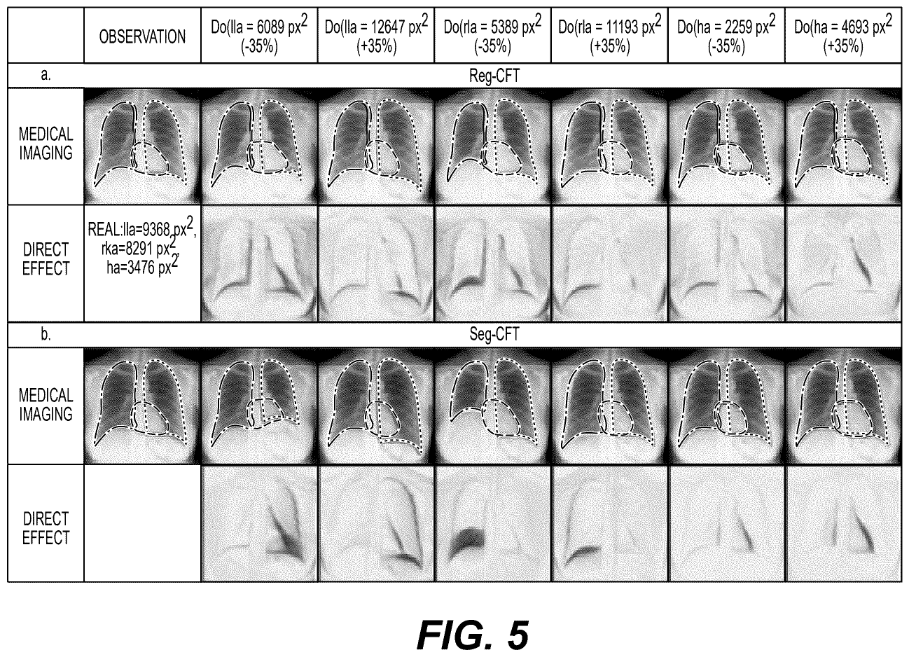

After Seg-CFT training, the measured outcome for the counterfactual image includes a target left lung area of 6,089 square pixels, a predicted left lung area of approximately 6,100 square pixels, a right lung area of approximately 8,290 square pixels, and a heart area of approximately 3,470 square pixels. As discussed with regard to FIG. 5 below, the visual difference in the counterfactual image is localized to the left lung boundary, with no global brightness shift and no unintended organ deformation.

The counterfactual image generated through this process represents a true counterfactual in the causal sense. The same latent image noise z is preserved, the same exogenous noise values u are preserved for non-intervened variables, and a single causal variable (left lung area) is intervened upon. The generated counterfactual image corresponds to the same patient, same scan, and same underlying anatomy, but with a smaller left lung. The counterfactual does not represent a different patient with smaller lungs, but rather the same individual patient under the hypothetical scenario in which the left lung area is reduced.

Visual examples depicted in FIG. 5 show that both Reg-CFT (a) and Seg-CFT (b) produce plausible counterfactual images, but Reg-CFT yields undesired effects outside the intervened structures. When intervening on left lung area, Reg-CFT may produce counterfactual images in which the right lung area and heart area are also modified, even though those structures were not targeted by the intervention. By contrast, Seg-CFT produces more locally coherent and targeted changes, resulting in more accurate interventions as reflected in the left lung area, right lung area, and heart area values predicted from the counterfactual images. The visual comparison suggests that Seg-CFT enables the DSCM to obtain a better understanding of which part of an image should be changed upon structure-specific interventions, producing modifications that are confined to the anatomical structure of interest while preserving other structures unchanged.

Quantitative effectiveness results of the above demonstrate that Seg-CFT achieves lower Mean Absolute Percentage Error (MAPE) compared to Reg-CFT and No-CFT across all intervention types. Without counterfactual fine-tuning (No-CFT), counterfactual images exhibit the highest MAPE values, indicating that the HVAE decoder ignores the conditioning variables and fails to produce images consistent with the specified interventions. Reg-CFT reduces MAPE compared to No-CFT, but Seg-CFT achieves the lowest MAPE for all intervened variables. The quantitative results highlight the importance of counterfactual fine-tuning to mitigate ignored counterfactual conditioning, and demonstrate that segmentor-guided supervision provides more effective guidance than regressor-based supervision for structure-specific interventions.

For the CCTA images, straightened curvilinear planar reformation (sCPR) was used to create 2D images from the longitudinal cross-section of the center-line of the left anterior descending (LAD) artery. All images were sampled at a resolution of 0.25×0.25 mm and cropped to 64×384 pixels.

Segmentation masks of the coronary lumen and plaque were also generated. To achieve this, 3D meshes of the coronary lumen and outer wall were sampled and rasterized in the 2D sCPR image plane, generating masks of the lumen, calcified plaque, and non-calcified plaque. A total of 18,433 CCTA images were used to generate samples, with 12,903 samples for training, 1,843 for validation, and 3,687 for testing. Three structure-specific variables were considered: calcified plaque area (CPA), non-calcified plaque area (NCPA), and lumen area (LA). For simplicity, it was assumed that these are independent of each other.

To run through an iteration of the training process for this evaluation, an intervention of increasing non-calcified plaque area by approximately 50 percent while preserving the lumen area and calcified plaque area unchanged is received. The DSCM for this application includes causal variables comprising non-calcified plaque area (NCPA) measured in square millimeters, calcified plaque area (CPA) measured in square millimeters, lumen area (LA) measured in square millimeters, and the image variable representing the sCPR image in pixels.

In an example iteration of the training process, an observed patient presents with a real sCPR image depicting the left anterior descending (LAD) artery. The observed values for this patient include non-calcified plaque area of 17.3 square millimeters, calcified plaque area of 3.5 square millimeters, and lumen area of 5.8 square millimeters. These values represent typical magnitudes from the CCTA dataset.

The abduction step infers the exogenous noise that produced the observed patient data. For scalar variables, exact inversion through the normalizing flows recovers the exogenous noise values. Notional noise values for this example include u_NCPA of +0.62, u_CPA of −0.11, and u_LA of +0.04. These noise values encode patient-specific plaque burden tendencies that characterize the individual patient's disease state. For the image latent, the HVAE encoder produces an approximate inference of the latent image noise z. A notional latent vector z=[−0.48, 1.03, 0.77, . . . ] captures patient-specific characteristics including vessel curvature, imaging noise, wall texture, and contrast dynamics.

The action step applies a causal intervention to answer the counterfactual question: “What if non-calcified plaque were worse?” The intervention specifies an increase in NCPA by approximately 50 percent, yielding a target value of 1.5×17.3=25.95 square millimeters. The formal do-operation do (NCPA=25.95) sets the non-calcified plaque area to the target value while no other noise values are changed.

The prediction step computes counterfactual parent values using the same abducted noise. The non-calcified plaque area is fixed by the do-operator at 25.95 square millimeters. The calcified plaque area is recomputed using the structural causal function with the abducted noise u_CPA, yielding approximately 3.5 square millimeters. The lumen area is similarly recomputed using the structural causal function with the abducted noise u_LA, yielding approximately 5.8 square millimeters. This computation encodes the causal assumption that increased soft plaque does not cause lumen collapse unless such a relationship is explicitly modeled in the causal graph.

The DSCM decoder generates a counterfactual image conditioned on the counterfactual parent values and the latent image noise z. Without Seg-CFT training, the model may exhibit failure modes when using Reg-CFT. With Reg-CFT, the model may learn that higher NCPA correlates with darker vessel appearance, reduced lumen brightness, or global intensity changes. The regressor-based supervision may allow the DSCM to satisfy the regressor by modifying global image properties rather than by adding plaque pixels in the anatomically correct location between the lumen and vessel wall.

During training with Seg-CFT, the frozen segmentor is applied to the generated counterfactual image. The segmentor produces probability maps for non-calcified plaque, calcified plaque, and lumen. Areas are computed by summing pixel-level probabilities within each structure. If the model produces a counterfactual image with measured non-calcified plaque area of 20.1 square millimeters, calcified plaque area of 4.6 square millimeters, and lumen area of 4.1 square millimeters, this represents a bad counterfactual. The non-calcified plaque area is too small relative to the target of 25.95 square millimeters, the calcified plaque area changed unintentionally from the original 3.5 square millimeters, and the lumen area shrank spuriously from the original 5.8 square millimeters.

The Seg-CFT loss applies to the intervened variable. The loss is computed as the absolute difference between the measured non-calcified plaque area and the target: 120.1−25.951=5.85 square millimeters. The calcified plaque area and lumen area are not rewarded for changing, as the loss function penalizes deviations from the target value for the intervened variable while the non-intervened variables are expected to remain stable. The gradient from this loss encourages the DSCM to add soft plaque pixels in the anatomically correct location to reduce the discrepancy between the measured and target non-calcified plaque area.

Through iterative training with Seg-CFT, the DSCM learns to produce anatomically coherent counterfactual images. To reduce the loss, the model learns to add plaque between the lumen and vessel wall, respect vessel geometry, preserve the calcified plaque region, and preserve the lumen cross-section. The segmentation-based supervision prevents the model from darkening the whole image, shrinking the lumen globally, or modifying calcified plaque texture, as such modifications would not reduce the Seg-CFT loss for the intervened variable while potentially increasing errors for non-intervened variables.

After Seg-CFT training converges, the measured outcome for the counterfactual image includes a target non-calcified plaque area of 25.95 square millimeters, an achieved non-calcified plaque area of approximately 25.7 square millimeters, a calcified plaque area of approximately 3.6 square millimeters, and a lumen area of approximately 5.7 square millimeters. The effectiveness of counterfactual generation is evaluated using mean absolute error in square millimeters for coronary artery interventions. The results demonstrate the lowest mean absolute error for the intervened variable (non-calcified plaque area) and minimal error on the non-intervened variables (calcified plaque area and lumen area).

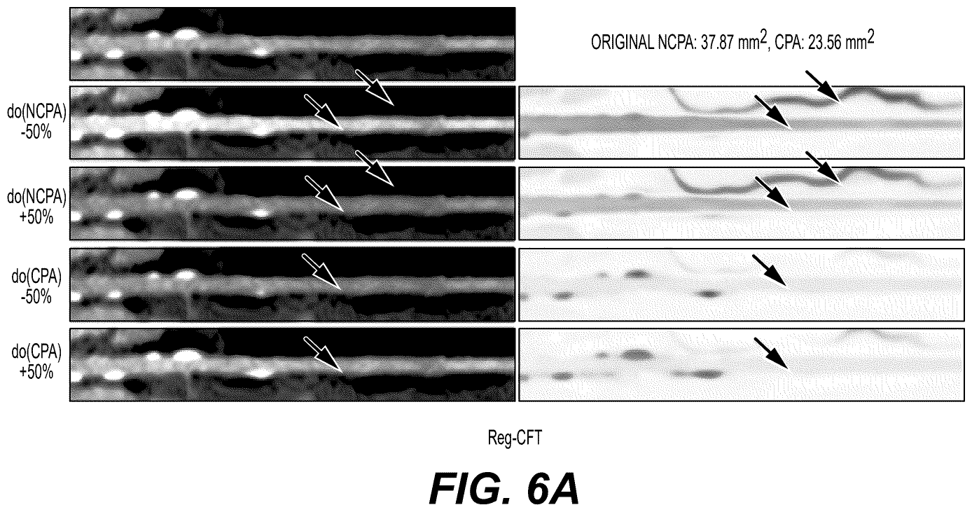

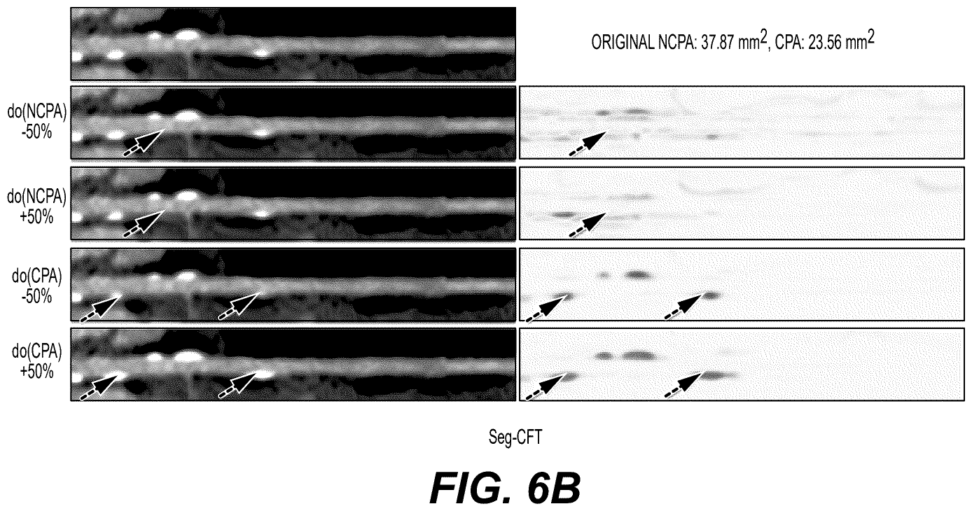

Referring now to FIG. 6, visual results comparing Reg-CFT (a) and Seg-CFT (b) for coronary artery interventions are depicted, namely standard regressor-based counterfactual fine-tuning (top) and segmentor-based counterfactual fine-tuning (bottom). The visual comparison demonstrates that Reg-CFT introduces unintended global effects across non-target structures. Interventions on non-calcified plaque area using Reg-CFT affect the global intensity of the lumen area, indicating that the regressor-based supervision allows the DSCM to satisfy the regressor through spurious correlations rather than anatomically meaningful changes. In contrast, Seg-CFT yields more localized effects, focusing modifications on the intervened structures. The plaque region thickens in the counterfactual image, the vessel wall expands outward to accommodate the increased plaque volume, the lumen remains stable without spurious shrinkage, and no global intensity shift is observed. The visual results demonstrate localized disease progression rather than a style change, with modifications confined to the anatomical structure of interest while preserving other structures unchanged.

The coronary artery disease example demonstrates advantages of Seg-CFT over Reg-CFT that are more pronounced than in the chest radiograph example. The coronary artery imaging application involves multiple adjacent structures (lumen, calcified plaque, and non-calcified plaque) with competing explanations for scalar measurements. The proximity of these structures and the potential for texture-based shortcuts make the coronary artery application particularly susceptible to the failure modes of regressor-based supervision. Seg-CFT succeeds in this challenging application by grounding scalar causal variables in pixel-level anatomy, forcing the DSCM to add plaque where plaque anatomically resides rather than producing images that merely appear more diseased through global intensity or texture modifications.

Quantitative effectiveness of the above show that Seg-CFT achieves the best performance, followed by Reg-CFT. Notably, Reg-CFT results in significantly higher MAE for un-intervened variables (indicated in gray text). This is likely due to the regressors learning spurious correlations, which subsequently affect DSCMs during fine-tuning. The visual results in FIG. 6 illustrate that Reg-CFT introduces unintended global effects across non-target structures. Note how interventions on NCPA affect the global intensity of the lumen area. In contrast, Seg-CFT yields much more localized effects, focusing specifically on the intervened structures. This demonstrates the advantage of incorporating segmentor-guidance in CFT.

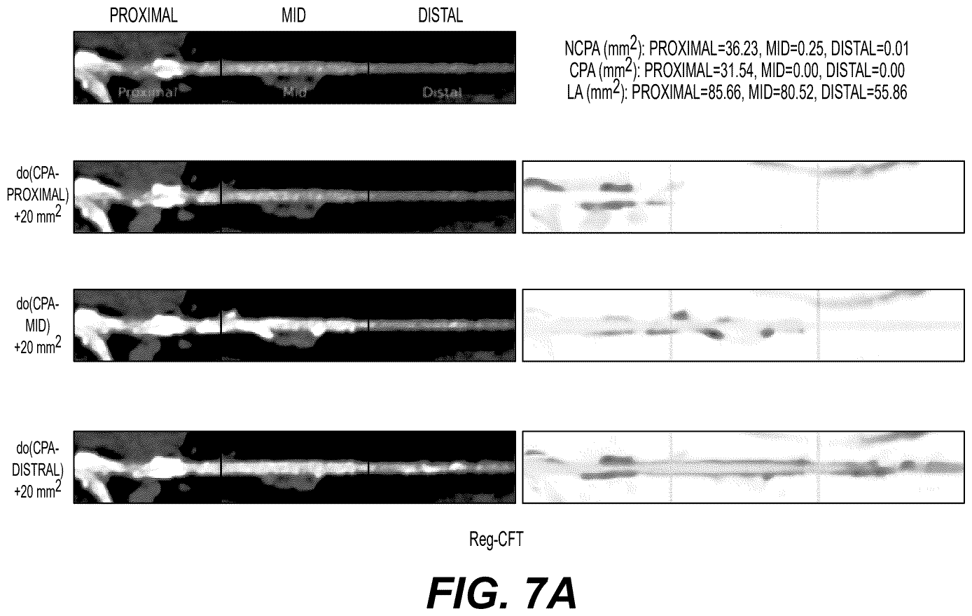

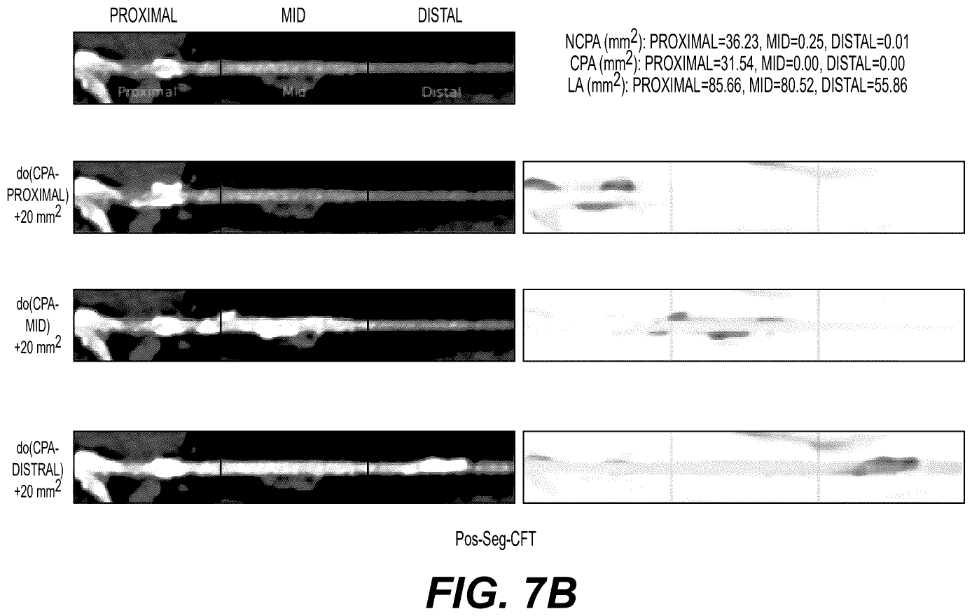

Referring now to FIG. 7, visual results comparing Reg-CFT (a) and Seg-CFT (b) for positional interventions on coronary arteries are depicted. FIG. 7 illustrates counterfactual images and corresponding difference maps for calcified plaque area (CPA) interventions of +20 square millimeters applied to the proximal, mid, and distal regions of the coronary vessel. Each row in FIG. 7 shows the counterfactual image on the left and the corresponding difference map on the right, with vertical dashed lines indicating the boundaries between the proximal, mid, and distal vessel segments.

The visual results for Reg-CFT depicted in FIG. 7 exhibit noticeable positional leakage. When interventions are applied to the mid region or the distal region using Reg-CFT, pronounced changes are observed in the proximal segment even though the proximal segment was not targeted by the intervention. This positional leakage indicates that regression-based supervision fails to preserve spatial independence between anatomical regions. The regressor-based approach allows the DSCM to satisfy the regressor through modifications that affect multiple regions simultaneously, rather than confining changes to the intended region of the intervention.

In contrast, the visual results for Pos-Seg-CFT depicted in FIG. 7 demonstrate well-localized and anatomically consistent modifications that are confined to the intended regions. When an intervention is applied to the proximal region using Pos-Seg-CFT, the modifications are concentrated within the proximal segment with minimal changes observed in the mid and distal segments. Similarly, interventions applied to the mid region produce modifications concentrated within the mid segment, and interventions applied to the distal region produce modifications concentrated within the distal segment. The spatial confinement of modifications to the intended regions demonstrates that Pos-Seg-CFT provides effective position-aware supervision during counterfactual fine-tuning.

The difference maps depicted in FIG. 7 further reveal the spatial characteristics of the counterfactual modifications. For Pos-Seg-CFT, the difference maps show spatially aligned responses that correspond to the intended region of intervention. The difference maps exhibit reduced off-target activations compared to Reg-CFT, indicating that Pos-Seg-CFT produces counterfactual images with fewer unintended modifications in regions that were not targeted by the intervention. The reduced off-target activations demonstrate improved precision in positional counterfactual generation, as the DSCM learns to localize changes to the anatomical region specified by the spatially targeted intervention.

The visual comparison between Reg-CFT and Pos-Seg-CFT in FIG. 7 demonstrates that positional segmentor-guided counterfactual fine-tuning substantially improves spatial localization of counterfactual modifications. By decomposing global structure-specific variables into regional measurements and providing region-specific supervision during fine-tuning, Pos-Seg-CFT enables the DSCM to produce counterfactual images in which modifications are anatomically coherent and spatially confined to the intended regions. The position-aware supervision provided by Pos-Seg-CFT encourages the generative model to produce spatially coherent modifications aligned with the intended positional interventions, reducing the entanglement between anatomical components that is observed with regression-based approaches.

Counterfactual image generation according to one or more aspects of this disclosure may be applied to disease progression modeling. Disease progression modeling involves simulating how anatomical structures or pathological features would appear under hypothetical scenarios representing different stages of disease. For coronary artery disease applications, counterfactual image generation may simulate how plaque burden would appear if the plaque burden increased in specific vessel segments. A clinician or researcher may specify a spatially targeted intervention that increases non-calcified plaque area or calcified plaque area within a proximal, mid, or distal region of a coronary vessel. The DSCM generates a counterfactual image depicting the same patient under the hypothetical scenario in which the plaque burden has progressed in the specified region. The generated counterfactual image may be used to visualize potential disease trajectories, assess the visual appearance of more advanced disease states, or train predictive models that anticipate disease progression based on current imaging findings. By preserving the latent noise variables that encode patient-specific characteristics, the counterfactual image represents the same individual patient with increased plaque burden rather than a different patient with more severe disease, enabling patient-specific disease progression modeling.

Counterfactual image generation may also be applied to data augmentation for training machine learning models. Data augmentation involves generating synthetic training examples that expand the diversity of a training dataset without requiring additional real patient data. Counterfactual image generation enables data augmentation with controlled anatomical variations, as the DSCM can generate synthetic images in which specific anatomical parameters are modified according to specified interventions while other characteristics remain consistent with the original patient data. For example, a training dataset for a lung segmentation model may be augmented by generating counterfactual chest radiographs in which lung areas are varied across a range of values, producing synthetic examples that span a broader distribution of anatomical configurations than present in the original dataset. The controlled nature of counterfactual generation ensures that the synthetic examples exhibit anatomically coherent variations rather than arbitrary image perturbations, as the DSCM has been trained to produce images that satisfy segmentation-derived measurements for the specified anatomical parameters. Data augmentation using counterfactual image generation may improve generalization of machine learning models by exposing the models to a wider range of anatomically plausible variations during training.

Counterfactual image generation may further be applied to bias mitigation in medical imaging datasets. Bias mitigation involves generating balanced datasets across different anatomical characteristics to reduce the influence of dataset imbalances on trained models. Medical imaging datasets may exhibit imbalances in the distribution of anatomical characteristics, such as overrepresentation of certain organ sizes, disease severities, or patient demographics. These imbalances may cause machine learning models trained on such datasets to exhibit biased performance, with reduced accuracy for underrepresented anatomical configurations. Counterfactual image generation enables bias mitigation by generating synthetic images that balance the distribution of anatomical characteristics across the dataset. For example, if a dataset contains predominantly patients with large lung areas, counterfactual image generation may produce synthetic images with smaller lung areas by applying interventions that reduce lung area parameters. The generated counterfactual images augment the dataset with examples representing underrepresented anatomical configurations, producing a more balanced training distribution. By generating balanced datasets across different anatomical characteristics, counterfactual image generation may reduce bias in trained models and improve equitable performance across diverse patient populations.

The methods described herein may extend to three-dimensional medical imaging including volumetric computed tomography (CT) scans and magnetic resonance imaging (MRI) scans. Three-dimensional medical imaging produces volumetric data that captures anatomical structures across multiple spatial dimensions, enabling visualization and analysis of complex three-dimensional anatomy. Extension of counterfactual image generation to three-dimensional imaging involves adapting the DSCM architecture to process volumetric data rather than two-dimensional images. The HVAE encoder and HVAE decoder may be modified to accept three-dimensional input volumes and produce three-dimensional output volumes, with convolutional operations extended to three spatial dimensions. The pre-trained segmentor used for segmentor-guided counterfactual fine-tuning may similarly be adapted to produce three-dimensional segmentation volumes that delineate anatomical structures throughout the volumetric data. Structure-specific parameters for three-dimensional imaging may include volumetric measurements such as organ volumes, plaque volumes, or lesion volumes, rather than area measurements used for two-dimensional imaging. Spatially targeted interventions for three-dimensional imaging may specify counterfactual changes to volumetric parameters within three-dimensional anatomical regions, enabling localized modifications throughout the volumetric data. Extension to three-dimensional medical imaging enables advanced counterfactual reasoning in high-dimensional data, supporting applications such as volumetric disease progression modeling, three-dimensional data augmentation, and bias mitigation across volumetric anatomical characteristics. Extension to further n-dimensional data, such as three-dimensional structures changing over time, is also contemplated.

The counterfactual generation process may explicitly modify additional characteristics beyond area measurements. While the examples described herein focus on area measurements as structure-specific parameters (such as lung area, plaque area, and lumen area), the counterfactual generation framework may be extended to modify other anatomical characteristics including shape, location, or texture. Shape modifications may involve interventions that alter the geometric configuration of anatomical structures, such as changing the aspect ratio of an organ, modifying the curvature of a vessel, or altering the morphology of a lesion. Location modifications may involve interventions that shift the spatial position of anatomical structures within the image, such as repositioning a lesion to a different anatomical location or translating an organ relative to surrounding structures. Texture modifications may involve interventions that alter the appearance characteristics of anatomical structures, such as modifying the intensity patterns within a tissue region, changing the contrast characteristics of a lesion, or altering the surface texture of an organ boundary. Extension to these additional characteristics involves defining appropriate scalar-valued or vector-valued parameters that capture the characteristics of interest, incorporating those parameters into the causal graph as causal parents of the image variable, and training the DSCM with segmentor-guided or measurement-guided counterfactual fine-tuning that provides supervision for the additional characteristics. The ability to explicitly modify shape, location, or texture within the counterfactual generation process provides greater flexibility in medical image synthesis, enabling generation of counterfactual images that explore a broader range of hypothetical anatomical configurations.

Machine readable storage including machine-readable instructions, when executed, to implement a method or realize an apparatus in any of the examples of the present application.

Various techniques, or certain aspects or portions thereof, may take the form of program code (i.e., instructions) embodied in tangible media, such as floppy diskettes, CD-ROMs, hard drives, a non-transitory computer readable storage medium, or any other machine-readable storage medium wherein, when the program code is loaded into and executed by a machine, such as a computer, the machine becomes an apparatus for practicing the various techniques. In the case of program code execution on programmable computers, the computing device may include a processor, a storage medium readable by the processor (including volatile and non-volatile memory and/or storage elements), at least one input device, and at least one output device. The volatile and non-volatile memory and/or storage elements may be a RAM, an EPROM, a flash drive, an optical drive, a magnetic hard drive, or another medium for storing electronic data. The eNB (or other base station) and UE (or other mobile station) may also include a transceiver component, a counter component, a processing component, and/or a clock component or timer component. One or more programs that may implement or utilize the various techniques described herein may use an application programming interface (API), reusable controls, and the like. Such programs may be implemented in a high-level procedural or an object-oriented programming language to communicate with a computer system. However, the program(s) may be implemented in assembly or machine language, if desired. In any case, the language may be a compiled or an interpreted language, and combined with hardware implementations.

It should be understood that many of the functional units described in this specification may be implemented as one or more components, which is a term used to more particularly emphasize their implementation independence. For example, a component may be implemented as a hardware circuit comprising custom very large scale integration (VLSI) circuits or gate arrays, off-the-shelf semiconductors such as logic chips, transistors, or other discrete components. A component may also be implemented in programmable hardware devices such as field programmable gate arrays, programmable array logic, programmable logic devices, or the like.

Components may also be implemented in software for execution by various types of processors. An identified component of executable code may, for instance, comprise one or more physical or logical blocks of computer instructions, which may, for instance, be organized as an object, a procedure, or a function. Nevertheless, the executables of an identified component need not be physically located together, but may comprise disparate instructions stored in different locations that, when joined logically together, comprise the component and achieve the stated purpose for the component.

Indeed, a component of executable code may be a single instruction, or many instructions, and may even be distributed over several different code segments, among different programs, and across several memory devices. Similarly, operational data may be identified and illustrated herein within components, and may be embodied in any suitable form and organized within any suitable type of data structure. The operational data may be collected as a single data set, or may be distributed over different locations including over different storage devices, and may exist, at least partially, merely as electronic signals on a system or network. The components may be passive or active, including agents operable to perform desired functions.

Reference throughout this specification to “an example” means that a particular feature, structure, or characteristic described in connection with the example is included in at least one embodiment of the present invention. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present invention may be referred to herein along with alternatives for the various components thereof. It is understood that such embodiments, examples, and alternatives are not to be construed as de facto equivalents of one another, but are to be considered as separate and autonomous representations of the present invention.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive, and the invention is not to be limited to the details given herein, but may be modified within the scope and equivalents of the appended claims.

Those having skill in the art will appreciate that many changes may be made to the details of the above-described embodiments without departing from the underlying principles of the invention. The scope of the present invention should, therefore, be determined only by the following claims.

We claim:

1. A computer-implemented method of counterfactual image generation, comprising:
obtaining medical imaging data including an image depicting anatomy of a patient;
determining latent parameter noise by applying a learned causal or structural relationship to one or more parameters associated with the medical imaging data;
determining a latent representation by applying an encoder network of a generative model to the image;
receiving one or more spatially targeted interventions, each spatially targeted intervention defining a counterfactual change to be applied to at least one parameter of the one or more parameters within a respective region of the anatomy;
generating a set of counterfactual parameters based on the latent parameter noise and the one or more spatially targeted interventions; and
generating a counterfactual image of the anatomy that includes the one or more spatially targeted interventions by applying a decoder network of the generative model to the set of counterfactual parameters and the latent representation.

2. The computer-implemented method of claim 1, wherein the encoder network is a Hierarchical Variational Auto Encoder (HVAE) encoder, the decoder network is a HVAE decoder, and the generative model is a Deep Structural Causal Model (DSCM) that includes a causal model of the learned causal or structural relationship of the one or more parameters.

3. The computer-implemented method of claim 1, wherein:
determining the latent representation includes applying the encoder network to the image and the one or more parameters; and
the generative model is fine-tuned with segmentation-guided loss to localize counterfactual changes to one or more targeted anatomical regions.

4. The computer-implemented method of claim 1, wherein the medical imaging data includes at least one of the one or more parameters.

5. The computer-implemented method of claim 1, further comprising:
determining at least one of the one or more parameters by applying an imaging analysis operation to the image.

6. The computer-implemented method of claim 5, wherein the imaging analysis operation includes segmenting the image using a pre-trained image segmentor.

7. The computer-implemented method of claim 6, wherein the pre-trained image segmentor is a same segmentor that was used to train the encoder network and decoder network.

8. The computer-implemented method of claim 1, wherein:
obtaining the medical imaging data includes receiving, via a Graphical User Interface (GUI) a selection of the patient and a selection of the image;
receiving the one or more spatially targeted interventions includes:
receiving, via the GUI, a natural language prompt describing the counterfactual change to be applied to the image; and
parsing the natural language prompt to extract the one or more spatially targeted interventions.

9. A computer-implemented method of training a causal generative model for counterfactual medical image generation, comprising:
obtaining training medical imaging data including images depicting anatomy of respective patients and associated causal parameters;
obtaining a causal generative model that includes (i) structural causal functions defining relationships between the associated causal parameters, and (ii) an encoder network and a decoder network;
iteratively, for one or more images in the training medical imaging data:
determining latent parameter noise by applying a current iteration of the structural causal functions to the associated causal parameters; and
determining a latent representation by applying a current iteration of the encoder network to an image of the one or more images;
obtaining one or more spatially targeted interventions, each spatially targeted intervention defining a counterfactual change to be applied to at least one parameter of the associated causal parameters within a respective region of the anatomy;
generating a set of counterfactual parameters based on the latent parameter noise and the one or more spatially targeted interventions;
generating a counterfactual image by applying the decoder network to the latent representation and the set of counterfactual parameters;
determining a loss of the generated counterfactual image by:
applying a pre-trained segmentor to the counterfactual image to obtain segmentation outputs for at least a portion of the anatomy;
determining measurements, from the segmentation outputs and corresponding to the set of counterfactual parameters, of the counterfactual image; and
determining the loss based on a difference between the measurements and the set of counterfactual parameters; and
updating the causal generative model based on the loss.

10. The computer-implemented method of claim 9, wherein:
individual parameters of the associated causal parameters are associated with respective regions of the anatomy; and
determining the measurement for an individual parameter includes masking out, weighting, or otherwise restricting any regions of the anatomy not associated with the parameter.

11. The computer-implemented method of claim 10, wherein determining the latent representation includes applying the current iteration of the encoder network to the image and the associated causal parameters.

12. The computer-implemented method of claim 9, wherein the pre-trained segmentor is frozen during updating of the causal generative model.

13. The computer-implemented method of claim 9, wherein obtaining the training medical imaging data includes determining the associated causal parameters by applying the pre-trained segmentor to the images and determining measurements, corresponding to associated parameters, of the images.

14. The computer-implemented method of claim 9, wherein obtaining one or more spatially targeted interventions includes selecting a randomized intervention for at least one of the associated causal parameters within a predefined range.

15. The computer-implemented method of claim 9, wherein obtaining one or more spatially targeted interventions includes:
obtaining a distribution of values for at least one of the associated causal parameters; and
selecting an intervention based on the distribution.

16. The computer-implemented method of claim 15, wherein the distribution is based on measurements, corresponding to the at least one of the associated causal parameters, of the images in the training medical imaging data.

17. A system for generating counterfactual images, comprising:
at least one memory storing:
instructions;
medical imaging data with an image depicting anatomy of a patient; and
a causal generative model that includes:
a learned causal or structural relationship between parameters associated with the medical imaging data;
an encoder network; and
a decoder network;
wherein the generative model is fine-tuned with segmentation-guided loss to localize counterfactual changes to one or more targeted anatomical regions; and
at least one processor operatively connected to the at least one memory and configured to execute the instructions to perform operations, including:
determining latent parameter noise of the image by applying the learned causal or structural relationship to the parameters;
determining a latent representation by applying the encoder network to the image;
receiving one or more spatially targeted interventions, each spatially targeted intervention defining a counterfactual change to be applied to at least one of the parameters within a respective region of the anatomy;
generating a set of counterfactual parameters based on the latent parameter noise and the one or more spatially targeted interventions; and
generating a counterfactual image of the anatomy that includes the one or more spatially targeted interventions by applying the decoder network of the generative model to the set of counterfactual parameters and the latent representation.

18. The system of claim 17, wherein determining the latent representation includes applying a Hierarchical Variational Auto Encoder (HVAE) to the image and the parameters.

19. The system of claim 17, wherein the medical imaging data includes at least one of the parameters.

20. The system of claim 17, wherein:
the operations further include determining at least one of the one or more parameters by applying a pre-trained image segmentor to the image; and
the pre-trained image segmentor is a same segmentor that was used to train the causal generative model.
